(19) United States
(12) Patent Application Publication
(10) Pub. No.: US 2026/0219276 A1
(43) Pub. Date: Jul. 30, 2026

(54) HYDROGEL BEAD-BASED PLATFORM FOR HIGH THROUGHPUT SCREENING OF IMMUNOMODULATORY PEPTIDES AND DESIGNER PEPTIDES FOR DENDRITIC CELLS/MACROPHAGE REPROGRAMMING

(71) Applicant: THE HONG KONG UNIVERSITY OF SCIENCE AND TECHNOLOGY, Hong Kong (CN)

(72) Inventors: Jin Teng CHUNG, Hong Kong (CN); Chi Ming Laurence LAU, Hong Kong (CN); Ying CHAU, Hong Kong (CN)

(21) Appl. No.: 19/143,622

(22) PCT Filed: Dec. 27, 2023
(86) PCT No.: PCT/IB2023/000790

§ 371 Date: Jun. 26, 2025

Related U.S. Application Data
(60) Provisional application No. 63/477,558, filed on Dec. 28, 2022.

(51) Int. Cl. G01N 33/58; C12N 15/10; G01N 33/50

(52) U.S. Cl. CPC G01N 33/582; C12N 15/1065; C12N 15/1096; G01N 33/5047; G01N 33/5055; G01N 2333/4703; G01N 2333/70578; G01N 2333/7158

(57) ABSTRACT

Provided is a novel bead-based platform that can be used to screen various immune active peptides and designer peptide candidates and combinations for their immunogenic or tolerogenic potential. More specifically, the platform uses peptides that are specified by parameters to increase their efficiency on immune cell differentiation.

### CROSS-REFERENCE TO RELATED APPLICATION

This application claims the benefit of U.S. Provisional Application Ser. No. 63/477,558, filed Dec. 28, 2022, which is hereby incorporated by reference in its entirety including any tables, figures, or drawings.

### REFERENCE TO SEQUENCE LISTING

The Sequence Listing for this application is labeled “HKUS185XPCT.xml” which was created on Dec. 21, 2023 and is 13,184 bytes. The entire content of the Sequence Listing is incorporated herein by reference in its entirety.

### BACKGROUND OF THE INVENTION

The immune system regulates disease progression, including cancer, tissue degeneration and regeneration, tissue transplants, aging, and autoimmune disorders. Immunomodulatory small molecule drugs, such as cyclosporine A, cyclophosphamide, levamisole, imiquimod, thiocarbamate, and penicillamine, have been synthesized and widely used in clinical settings to control infection and protect immune functions [1]-[4]. Nonetheless, these drugs can exhibit toxicity and adverse effects, limiting their long-term use for the treatment of chronic diseases. On the other hand, naturally derived immunomodulatory proteins or peptides show higher biocompatibility, implicating their potential in immunotherapy, particularly as adjuvants for the vaccine development against complex diseases, including cancer, autoimmune disorders, and degenerative disorders. With emerging sequencing technology and data science, numerous novel antigens and epitopes have been identified for use in subunit vaccines against complex diseases. However, the clinical performance has been largely limited by the poor immunogenicity/tolerogenicity of the antigens and rapid clearance from the body before inducing potent and durable immune responses and memory. Therefore, antigens are often co-delivered with adjuvants to improve their immune outcomes.

There are numerous adjuvants used in developing vaccine formulations; they can be naturally-occurring or chemically designed and synthesized. There are multiple naturally-occurring immune active resources, including proteins and peptides, isolated from plants and microbes. Additionally, some marine-derived proteins have demonstrated immunomodulatory properties [5], [6]-[7]. These proteins are classified as antimicrobial peptides (AMPs), host-defense peptides (HDPs) or innate defense regulators (IDRs) (see, for example, worldwide website: dbaasp.org/home). These proteins exhibit vast structural diversity.

Immune active proteins and peptides can be used to develop biomaterial-based adjuvants to improve the immunogenicity or tolerogenicity of the antigens in vaccine development and regenerative medicine. Nonetheless, there are immune active proteins that remain unexplored for their potential in vaccine development. Thus, there remains a need for additional products and methods that can be used for efficient investigation of the effects of various proteins and peptides on immune cell differentiation.

### BRIEF SUMMARY OF THE INVENTION

The subject invention relates to a novel, bead-based platform that can be used to screen various immune active peptides, designer peptide candidates, or combinations thereof for immunogenic or tolerogenic potential. In certain embodiments, the subject platform can be used to evaluate phenotypic changes in innate immune cells in vitro. In certain embodiments, the bead-based platform comprises hydrogel microparticles (HMPs) with variable sizes, stiffnesses, and degradability. In certain embodiments, these parameters are fine-tuned to investigate the biophysical effects of peptides on immune cell differentiation.

In certain embodiments, the subject bead-based platform can be used in high-throughput construction of a peptide and peptidomimetics library for novel drug design, synthesis, and screening. In certain embodiments, the subject bead-based platform can be the solid phase for peptide conjugation and screening for immune activity. In certain embodiments, the subject bead-based platform can serve as solid support for peptide immobilization, enzyme and substrate storage for high throughput screening (HTS) assays and antigen delivery platforms in vaccine formulations.

In certain embodiments, the subject bead-based platform can be used to test the effects of different combinations of immune-active peptides, presentation ratios, and densities on innate immune cell maturation phenotypes. In certain embodiments, the subject bead-based platform comprises four core elements: control of hydrogel core preparation, control of combinatorial peptide display, compatibility with high throughput microfluidics workflows (e.g., barcoding and reading, multiplexing, and sequencing, data analysis and interpretation), and compatibility with a diverse range of HTS assays (e.g., cell-based, affinity, or catalytic) (FIG. 1).

In certain embodiments, the subject bead-based platform can be used as a vaccine delivery vehicle and depot for the prolonged release of antigens and subunits in vivo. In certain embodiments, the subject bead-based platform can measure the host immune responses towards immunogenic activation or tolerance against the antigen of interest, thereby enabling the rapid development of various vaccine formulations with greater potency in immune protection and memory.

Innate immune cells, such as, for example, dendritic cells (DCs) and macrophages, are plastic; they polarize to pro-inflammatory (immunogenic/M1) and anti-inflammatory (tolerogenic/M2) phenotypes. In certain embodiments, the subject bead-based platform can be used to test peptides as adjuvants for the development of vaccines against complex diseases, such as, for example, cancer, autoimmune and degenerative disorders, by evaluating the effects of the peptides on innate immune cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. The four elements of proposed high throughput screening workflow and respective design parameters.

FIGS. 2A-2E. HMP preparations and physical characterizations. FIG. 2A Droplet-based microfluidics synthesis of HMPs. FIG. 2B SPG membrane extrusion of HMPs. FIG. 2C Microscope images of HMPs prepared with different methods, bulk emulsion, microfluidics, straining with 50 μm cell strainer and SPG extrusion. Images were taken under 20× magnification, scale bar represents 50 μm. The table shows the mean particle diameter±s.d. FIG. 2D The size distribution of HMPs prepared with bulk emulsion and FIG. 2E microfluidics, SPG extrusion and straining.

FIGS. 3A-3D. FIG. 3A JAWSII and RAW264.7 interactions with HMPs. JAWSII were co-cultured with FITC-conjugated HMPs (green) prepared with different methods in giving size range of 10 μm, 40 μm and 100 μm. Cells were stained with Hoechst 33342 (blue) and Lysotracker-Red (red). Co-localization of HMP and lysosomes gave yellow signals. Images were taken under fluorescence confocal microscopy at 20× magnification. Scale bar represents 50 μm. FIG. 3B Surface marker expression of CCR7 (lymph node homing marker), CD40 (co-stimulatory marker) of JAWSII were analyzed with flow cytometry to examine the cell activation status. Cells were cultured with HMPs of various size prepared at 30% wt/V and FIG. 3C various polymer concentrations of HMP at 40 μm for 96 hours (N=4, * is p<0.05 in comparison to other groups). FIG. 3D RAW264.7 macrophage were cultured with HMPs of various size at 30% wt/V. M1 marker, CCR7 and M2 marker, CD206 were examined with flow cytometry (N=4, * is p<0.05 in comparison to 40 μm).

FIGS. 4A-4H. MALDI-TOF and chemical structures of peptide species no. 1 (FIG. 4A), 2 (FIG. 4B), 3 (FIG. 4C), 4 (FIG. 4D), 5 (FIG. 4E), 6 (FIG. 4F), 7 (FIG. 4G), and 8 (FIG. 4H).

FIG. 5. The chemical structures of peptide species 9 to 19. The dipeptide/amino acid region is shaded in blue.

FIGS. 6A-6H. MALDI-TOF of peptide species 9 (FIG. 5A), 10 (FIG. 6B), 11 (FIG. 6C), 12 (FIG. 6D), 13 (FIG. 6E), 14 (FIG. 6F), 15 (FIG. 6G), and 16 (FIG. 611).

FIGS. 7A-7D. The response of JAWSII NF-κB NanoLuciferase® (Promega, Madison, WI) reporter system. FIG. 7A Viability of cells are measured with MTT assay. FIG. 7B NF-κB signaling activity are detected by the luminescence generated from the catalytic activity of luciferase. (N=3, * is p<0.05 in comparison to 0, non-decorated HMP). FIGS. 7C-7D Pooled cells loaded into microwells.

### BRIEF DESCRIPTION OF THE SEQUENCES

SEQ ID NO: 1: mLL-37 peptide
SEQ ID NO: 2: mlDR-1002 peptide
SEQ ID NO: 3: mQ peptide
SEQ ID NO: 4: mTLR2-1 peptide
SEQ ID NO: 5: C-SKKKK peptide
SEQ ID NO: 6: C-GGCSKKKK peptide
SEQ ID NO: 7: C-RGPPP peptide
SEQ ID NO: 8: LL-37 peptide
SEQ ID NO: 9: W1 site primer
SEQ ID NO: 10: PE1 site primer
SEQ ID NO: 11: Poly T region
SEQ ID NO: 12: peptide linker
SEQ ID NO: 13: peptide linker
SEQ ID NO: 14: peptide linker

### DETAILED DISCLOSURE OF THE INVENTION

The subject invention provides a novel, bead-based platform that can be used as a vaccine delivery vehicle. In certain embodiments, the subject bead-based platform can be used in methods of screening various immune active peptides, designer peptide candidates, or combinations thereof for immunogenic or tolerogenic potential by evaluating the phenotypic changes in innate immune cells, such as, for example, dendritic cells and macrophages, in vitro.

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting of” and “consists of” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with gene, cDNA, and mRNA encoded by a gene.

The term “hybridizes with” indicates that the two sequences are sufficiently complementary to each other to allow hybridization between the two sequences. Sequences that hybridize with teach other can be perfectly complementary but can also have mismatches to a certain extent. Depending upon the stringency of hybridization, a mismatch of about 5% to about 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred.

In this application, the terms “polypeptide”, “peptide”, and “protein” are used interchangeably herein to refer to a polymer of amino acids. The terms apply to amino acid polymers in which one or more amino acid residues are artificial chemical mimetics of corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the term “amino acid” refers to standard nomenclature, amino acid residue as denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, “innate defense regulators” or “IDRs” are synthetic immunomodulatory versions of natural host defense peptides.

As used herein, “antimicrobial peptides”, “AMPs”, “host-defense peptides”, or “HDPs” are a class of small peptides that inhibit or reduce the growth of a microorganisms, fungi, cancerous cells, or viruses.

As used herein, “innate defense regulators” or “IDRs” are synthetic immunomodulatory peptides, which can be versions of natural host-defense peptides.

As used herein, “adjuvants” refer to any substance that can enhance the subject's immune response to an antigen.

As used herein, “antigen” refers to any toxin or other foreign substance that can induce an immune response in the subject.

As used herein, the term “hydrogel” refers to a substance formed when an organic polymer (natural or synthetic) is cross-linked via covalent, ionic, or hydrogen bonds to create a three-dimensional open-lattice structure which entraps water molecules to form a gel.

“Subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate (e.g., Drosophila spp.).

“Treatment”, “treating”, “palliating” and “ameliorating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying cancer such that an improvement is observed in the patient, notwithstanding that the patient may still be afflicted with the cancer.

As used herein, the term “cancer” refers to the presence of cells possessing abnormal growth characteristics, such as uncontrolled proliferation, immortality, metastatic potential, rapid growth and proliferation rate, perturbed oncogenic signaling, and certain characteristic morphological features.

The term “effective amount” or “therapeutically effective amount” refers to that amount of a peptide or protein presented on a hydrogel microbead described herein that is sufficient to affect the intended application, including but not limited to disease treatment. The therapeutically effective amount may vary depending on the intended application (in vitro or in vivo) or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., innate immune cells. The specific dose will vary depending on the particular compounds chosen, the dosing regimen to be followed, whether it is administered in combination with other compounds, timing of administration, the tissue to which it is administered, and the physical delivery system in which it is carried.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

### Hydrogel Bead-Based Platform

The subject hydrogels are formed from polymers that provide chemically defined and versatile support for peptides or proteins. The properties of the hydrogels can be changed by varying polymer structures, polymer molecular weight, and cross-linking reaction conditions. In certain embodiments, the components of the hydrogel can be provided either separately or combined, in dry form, such as powders, or in solution form. The hydrogel forms when cross linking conditions are applied. For example, the precursor components of the hydrogel are dissolved in a liquid, such as water, and the solution temperature is increased to induce cross linking and form the hydrogel.

In certain embodiments, the subject bead-based, hydrogel platform can comprise a polysaccharide, such as, for example, cyclodextrin, alginate, dextran or any combination thereof or a polyether, such as, for example, polyethylene glycol. In preferred embodiments, the hydrogel comprises dextran. In certain embodiments, each hydroxyl group can be chemically modified or at least 1, 2, 3, 4 5, 6, 7, 8, 9, 10, or more hydroxyl groups of the polysaccharide can by chemically modified. In certain embodiments, the polysaccharide or polyether is modified with various functional groups, such as, for example, alkyl halide, carboxylate, alcohol, epoxide or vinyl sulfone groups (-VS), and, optionally, subsequently with DTT to create, for example, free thiol groups (—SH); modified with carbazate; or oxidized into aldehydes. In certain embodiments, the degree of modification can be measured by, for example, 1H-NMR spectroscopy and/or colorimetric methods, such as, for example Ellman's assay. In certain embodiments, the degree of modification can be about 1% to about 50%. In certain embodiments, the degree of modification can be altered by changing the reaction time and/or the degree of oxidation by changing the amount of the oxidant, such as, for example, hypochlorite, hydrogen peroxide, TEMPO-NaClO—NaBr, or NaIO₄. In certain embodiments, each about 1 g of the polysaccharide or polyether can be reacted with about 0.3 mmol to about 6.2 mmol of the oxidant.

In certain embodiments, the modified polysaccharide can be crosslinked to form a hydrogel polymer network using hydrogel precursor A and hydrogel precursor B. In certain embodiments, hydrogel precursor A and hydrogel precursor B can be mixed at a ratio of about 1:1 and 1:10, about 1:1 and about 1:9, about 1:1 and about 1:8, about 1:1 and about 1:7, about 1:1 and about 1:6, about 1:1 and about 1:5, about 1:1 and about 1:4, about 1:1 and about 1:3, about 1:1 and about 1:2, about 1:1 and about 1:1.5. In certain embodiments, hydrogel precursor B and hydrogel precursor A can be mixed at a ratio of about 1:1 and 1:10, about 1:1 and about 1:9, about 1:1 and about 1:8, about 1:1 and about 1:7, about 1:1 and about 1:6, about 1:1 and about 1:5, about 1:1 and about 1:4, about 1:1 and about 1:3, about 1:1 and about 1:2, about 1:1 and about 1:1.5. In certain embodiments, hydrogel precursor A can be dextran modified with vinyl sulfone and precursor B can be dextran modified with a thiol group (Table 1; row 1), hydrogel precursor A can be dextran modified with a thiol group and hydrogel precursor B can be dextran modified with vinyl sulfone (Table 1; row 2); hydrogel precursor A can be oxidized dextran and hydrogel precursor B can be dextran modified with carbazate group (Table 1; row 3).

In certain embodiments, the polysaccharide can have a weight per volume concentration in the hydrogel microparticle of about 5.0% to about 50%, about 7.5% to about 30%, or about 15%.

In certain embodiments, the modified polysaccharide can be bound to at least one peptide, such as, for example, an IDR peptide or a muramyl peptide. In certain embodiments, the peptide is LL-37 (LLGDFFRKSKEKIGKEFKRIVQRIKDFLRNLVPRTES; SEQ ID NO: 8) or a derivative thereof, such as, for example, FKRIVQRIKDFLR (SEQ ID NO: 1); IDR-1002 (VQRWLIVWRIRK SEQ ID NO: 2); QHREDGS (SEQ ID NO: 3); CSKKKK (SEQ ID NO: 4); SKKKK (SEQ ID NO: 5); GGCSKKKK (SEQ ID NO: 6); RGPPP (SEQ ID NO: 7); GG; GA; GV; GL; GF; GC; GD; GK; S; T; or Y.

In certain embodiments, the peptide has a N-terminus modification, such as, for example, maleimide hexanoate, cysteine, alkyne, azide-modified peptide, or 3-[(3-acrylamidepropyl)dimethylammonio]propanoate (ac). In certain embodiments, the peptide has a C-terminus modification, such as, for example, carboxamides, ester, alcohols, amides, alkyl amides, p-Nitroanilide, cyclic amides and modifications with dyes. In certain embodiments, the side chain hydroxyl group of the peptide can be modified with a zwitterionic moiety, such as, for example, phosphorylcholine, sulfobetaine, carboxylbetaine, or phosphoserine. In certain embodiments, the peptide can be modified with a linker, such as, for example, a flexible linker, such as, for example (G)n in which n is the number of Glycine residues (e.g., a G-G linker) and n can be 2 to 6; rigid linkers, such as, for example, (EAAK)n (SEQ ID NO: 12) in which the n can be 1 to 5, or (XP)n, in which the n can be 1 to 10; cleavable linkers, such as, for example, a disulfide or protease sensitive sequence, such as, for example GTAR (SEQ ID NO: 13) or DRIR (SEQ ID NO: 14); or C₆ (NH₂—C₅H₁₀—COOH) between the peptide and peptide N-terminus adapter. In certain embodiments, the peptides can be produced via automated microwave-facilitated solid phase peptide synthesis (SPPS) or classical solution phase synthesis. In certain embodiments, a maleimide hexanoate (m) can be added to a peptide, such as, for example, an Fmoc-deprotected peptide, via condensation between a primary amine and a carboxylate during SPPS. In certain embodiments, a cysteine residue (Cys) can be added to a peptide during SPPS to provide free thiols or amines for coupling a peptide with free vinyl sulfone groups or aldehyde, respectively. In certain embodiments, a cationic adapter, 3-[(3-acrylamidepropyl)dimethylammonio]propanoate (ac) can be added at the N-terminus of a peptide via condensation between carboxylate and a primary amine group. In certain embodiments, a hydroxyl-side chain of an amino acid residue, such as, for example, Ser, Tyr or Thr can be coupled to a Wang-resin, rink amide resin, or 2-Chlorotrityl chloride (CTC) resin in a solvent, such as, for example, Dimethylformamide (DMF), dichloromethane (DCM), N-methyl-2-pyrrolidone (NMP), or dimethylacetamide (DMAc) then a hydroxyl protective group (e.g., tBoc on the amine of lysine or tryptophan, tBu on the —OH group of serine, threonine, or tyrosine; Trt on cysteine, histidine, asparagine, or glutamine; or Pbf on arginine) in an acid and a solvent (e.g., DMF, DCM, NMP, or DMAc) was added to the amino acid residue, followed by the addition of 2-chloro-1,3,2-dioxaphospholane 2-oxide in a solvent and then N-[3-(Dimethylamino)propyl]methacrylamide dissolved in acetonitrile in order to modify the amino acid residue with a zwitterionic moiety, such as, for example phosphorylcholine or sulfobetaine from the modifications with 3-[[2-(Methacryloyloxy)ethyl]dimethylammonio]propane-1-sulfonate, 4-[[2-(Methacryloyloxy)ethyl]dimethylammonio]butane-1-sulfonate, 3-[[2-(Acryloyloxy)ethyl]dimethylammonio]propane-1-sulfonate, 3-[(3-Methacrylamidopropyl)dimethylammonio]propane-1-sulfonate, 4-[(3-Methacrylamidopropyl)dimethylammonio]butane-1-sulfonate, 3-[(3-Acrylamidopropyl)dimethylammonio]propane-1-sulfonate.

### Methods of Making the Hydrogels

In certain embodiments, a hydrogel microparticle can be formed using the hydrogel precursors in a process of bulk emulsion, cell straining, Shirasu porous glass (SPG) extrusion with a pore size of about 0.1 μm to about 50 μm, and/or microfluidics.

In certain embodiments, bulk emulsion, cell straining, and SPG extrusion were performed in water-in-oil emulsions to control the size and distribution of the resulting hydrogel microparticle. In certain embodiments, the continuous phase (oil phase) comprises about 75% to about 98%, about 80% to about 98%, about 85% to about 98%, about 90% to about 98%, or about 95% to about 98% v/v n-heptane, mineral oil, cyclohexane, or hexane and a surfactant, such as, for example, Span 80, Tween 80, saponin, Triton-X, sorbitan monooleate, polyoxyethylene, or any combination thereof. In certain embodiments, two surfactants can be used at a ratio of about 250:1, about 200:1, about 150:1, about 100:1, about 50:1, about 25:1, about 10:1, about 3:1, about 1:1. In preferred embodiments, the surfactant of the continuous phase comprises about 1.5% Span 80 and about 0.5% Tween 80. In certain embodiments, the discontinuous phase (aqueous phase) comprises at least one hydrogel precursor prepared in a buffer, such as, for example, phosphate buffer and/or PBS, with a controlled pH of about 5.5 to about 8.0.

In certain embodiments, microfluidics assisted droplet synthesis was performed using a continuous phase. In certain embodiments, the continuous phase comprises about 75% to about 98%, about 80% to about 98%, about 85% to about 98%, about 90% to about 98%, or about 95% to about 98% v/v n-heptane, mineral oil, cyclohexane, or hexane and a surfactant, such as, for example, Span 80, Tween 80, saponin, Triton-X, sorbitan monooleate, polyoxyethylene, or any combination thereof. In certain embodiments, two surfactants can be used at a ratio of about 250:1, about 200:1, about 150:1, about 100:1, about 50:1, about 25:1, about 10:1, about 1:1. In preferred embodiments, the surfactant of the continuous phase comprises about 75% to about 95%, about 80% to about 95%, about 85% to about 95%, about 90% to about 95%, or about 95% v/v mineral oil, n-heptane, cyclohexane, or hexane and a surfactant, such as, for example, Span 80, Tween 80, or a combination thereof. In certain embodiments, two surfactants can be used at a ratio of about 250:1, about 200:1, about 150:1, about 100:1, about 50:1, about 25:1, about 10:1, about 1:1 to create a hydrogel microparticle. In certain embodiments, the discontinuous phase comprises at least one hydrogel precursor in a solvent.

In certain embodiments, the resulting hydrogel microparticle has a mean diameter of about 100.0 μm to about 200.0 μm, about 114.6 μm to about 159.2 μm, or about 136.9 μm when prepared with microfluidics. In certain embodiments, the resulting hydrogel microparticle has a mean diameter of about 0.1 μm to about 200.0 μm, about 0.9 μm to about 119.5 μm, or about 136.9 μm when prepared with bulk emulsion. In certain embodiments, a cell strainer with a diameter of about 1 μm to about 200 μm can be used to prepare the hydrogel microparticle. In certain embodiments, the resulting hydrogel microparticle has a mean diameter of about 10 μm to about 50.0 μm, about 17 μm to about 46.4 μm, or about 31.7 μm when prepared with bulk emulsion and then filtered with 50 μm cell strainer. In certain embodiments, the resulting hydrogel microparticle has a mean diameter of about 1 μm to about 15.0 μm, about 3.3 μm to about 9.7 μm, or about 6.0 μm when prepared with an SPG membrane extrusion.

### Methods of Use

The subject invention relates to a novel, bead-based platform that can be used to screen various immune active peptides, designer peptide candidates, or combinations thereof for immunogenic or tolerogenic potential. In certain embodiments, the subject platform can be used to evaluate phenotypic changes in innate immune cells in vitro. In certain embodiments, the bead-based platform comprises hydrogel microparticles (HMPs) with variable sizes, stiffnesses, and degradability. In certain embodiments, these parameters are fine-tuned to investigate the biophysical effects of peptides on immune cell differentiation.

In certain embodiments, the HMP can be co-cultured with an innate immune cell, such as, for example, an immature dendritic cell line (e.g., JAWSII) or a macrophage cell line (e.g., RAW Blue™, InvivoGen, San Diego, CA), to investigate the effect of HMP size and mechanical stiffness on cell-HMP interactions. In certain embodiments, the interaction between the HMP and the immune cell can be visualized using a probe conjugated to the HMP, such as, for example, a fluorescent probe (e.g., a fluorescein (FITC), rhodamine, cyanine, BODIPY, or naphthalimide probe). In certain embodiments, lysosomes and late endosomal compartments can be visualized.

In certain embodiments, the activity of NFκB, CCR7, CD40, or any combination thereof can be measured in the immune cell co-cultured with the HMP. In certain embodiments, the peptide has immunogenic activity and/or tolerogenic activity when the NFκB activity, the CCR7 activity, or the CD40 activity is higher than the NFκB activity, the CCR7 activity or the CD40 activity of an immune cell that has not been co-cultured with the HMP. In certain embodiments, the NFκB activity, the CCR7 activity, or the CD40 activity is about 0.01% to about 500% higher than the NFκB activity, the CCR7 activity or the CD40 activity of an immune cell that has not been co-cultured with the HMP.

In certain embodiments, the subject bead-based platform can be used in high-throughput construction of a peptide and peptidomimetics library for novel drug design, synthesis, and screening. In certain embodiments, a combination of peptide and peptidomimetics are conjugated to the residual functional groups on HMPs via click chemistry. In certain embodiments, the peptide density can be adjusted by modifying the degree of modifications of dextran or any other polysaccharide or polyether precursors prior to HMP formation. Conjugation occurs in aqueous solvents containing the HMP suspension and a peptide mixture. In certain embodiments, unreacted peptides are removed with excess solvent washing. In certain embodiments, cargo loading, such as, for example, enzyme substrates, antigens, nucleic acids for barcoding and reading, is performed during the HMP synthesis and prior to peptide tagging.

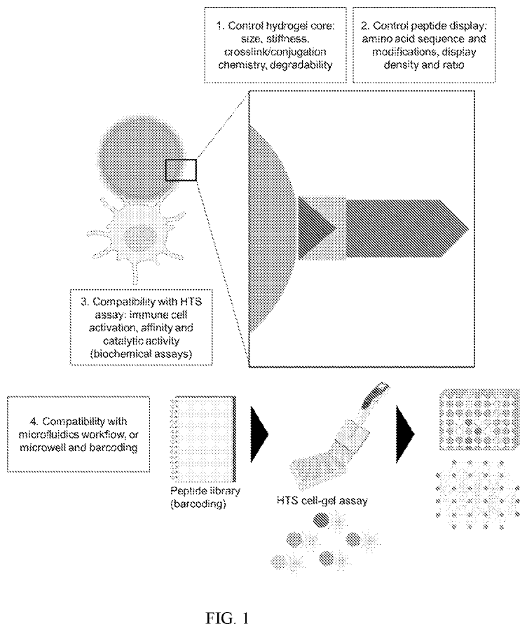

In certain embodiments, the subject bead-based platform can be the solid phase for peptide conjugation and screening for immune activity. In certain embodiments, the subject bead-based platform can serve as solid support for peptide immobilization, enzyme and substrate storage for high throughput screening (HTS) assays and antigen delivery platforms in vaccine formulations (e.g., cell-based, affinity, or catalytic) (FIG. 1). In certain embodiments, for cell-based activity, HMP are co-cultured with primary cells and reporter cell lines, such as, for example, RAW264.7 with NF-kB luciferase reporter gene, to probe cell activity in response to peptide ligand and receptor activity. In certain embodiments, for affinity, receptor and peptide binding, affinity can be measured with immunofluorescence assay, affinity chromatography, surface plasmon resonance, and isothermal titration calorimetry. In certain embodiments, the effect of the conjugated peptide mixture on HMP on enzyme catalytic activity from bio-samples can also be measured.

In certain embodiments, the subject bead-based platform can be used to test the effects of different combinations of immune-active peptides, presentation ratios, and densities on innate immune cell maturation phenotypes. In certain embodiments, immune active peptides and combinations thereof can be immobilized on HMPs via click chemistry. In certain embodiments, the presentation ratios depend on peptide mixing; whereas, the densities of peptide presentation depend on the degree modification of dextran or any other polysaccharide or polyether precursors and residual functional groups after HMP formation to conjugate the peptides.

In certain embodiments, the HMP can be co-cultured with a cell. In certain embodiments, an indexing primer can be added to the HMP and the cell. The indexing primer can comprise a barcode sequence and further comprise a sequence that hybridizes with an mRNA transcript of a gene in the cell. In certain embodiments, indexing primers can be about 20 to about 30 nucleotides in length. Exemplary indexing primers include: W1 site: AAGGCGTCACAAGCAATCACTC (SEQ ID NO: 9) and PE1 site: GAAGAGCGTCGTGTAGGGAAAGAG (SEQ ID NO: 10). In certain embodiments, a PolyT region, such as, for example, TTTTTTTTTTTTTTTTTTTV (SEQ ID NO: 11), can be added to the indexing primer to hybridize with the polyA tail of mRNAs in the cell.

In certain embodiments, a barcode sequence is a short unique sequence (about 4 to about 16 nucleotides) that is used to link the sequencing data to a respective sample. In certain embodiments, the mRNA transcript can be reverse transcribed to yield a cDNA sequence; the cDNA sequence can be amplified by polymerase chain reaction to yield an amplified cDNA sequence; and the amplified cDNA sequence can be sequenced.

In certain embodiments, the subject bead-based platform can be used as a vaccine delivery vehicle and depot for the prolonged release of antigens and subunits in vivo. In certain embodiments, the subject bead-based platform can measure the host immune responses towards immunogenic activation or tolerance against the antigen of interest, thereby enabling the rapid development of various vaccine formulations with greater potency in immune protection and memory. In certain embodiments, hydrogels and/or HMP vaccines can be administered to a subject via a subcutaneous, intramuscular, or intratumoral route. In certain embodiments, the HMP can be administered more than one time, such as, for example, at least 2, 3, 4, 5, 6, 7, 8, 9, 10 or more times. In certain embodiments, doses of the HMP can be separated by about 6 hours, about 12 hours, about 1 day, about 1 week, about 2 weeks, about 1 month, about 3 months, about 6 months, about 1 year, about 2 years, about 3 years, about 5 years, or about 10 years. In certain embodiments, for in vivo response monitoring, humoral immunity can be measured, such as, for example, antigen specific IgG1 and/or IgG2 levels in blood plasma can be measured. In certain embodiments, systemic cytokine levels, such as, for example, serum IL-6, IL-1β, IFN-γ, TNF-α, IL-10, and/or BAFF, can be monitored post-vaccination.

In certain embodiments, cellular immunity, such as, for example, T-cell population and subtype profiling in secondary lymphoid organs, such as, for example, lymph node and spleen, can be measured. In certain embodiments, cytolytic activity of CD8-T and NK cells can also be evaluated to examine the vaccine potency. In certain embodiments, professional antigen presenting cells, such as, for example, dendritic cells, macrophages, microglia and/or B-cells, are targeted by the subject HMPs.

In certain embodiments, the target of the subject immunogenic HMP vaccine includes cancer, solid tumors, and infectious disease. In certain embodiments, tolerogenic HMP vaccines can be used against autoimmune disorders, such as, for example, type-1 diabetes, rheumatoid arthritis, or multiple sclerosis.

In some embodiments, the schemes outlined above can provide a method of preparing a vaccine delivery vehicle. In some embodiments, the subject invention provides a high throughput screening (HTS) system and workflow to screen and mine for natural peptide-derivatives or designer peptides ligands manifesting immunogenic/tolerogenic properties appropriate for vaccine delivery.

### Materials and Methods

### Control of Hydrogel Core

Herein we adopted dextran (DX) to prepare HMP hydrogel core. DX is a bacterial polysaccharide, consisting of α-1,6-linked D-glucopyranose residues. DX is widely used in biomedical applications due to its biocompatibility, low cost of production and versatility in chemical modifications. The hydroxyl groups can be modified chemically with various functional groups for the drug delivery device development. The hydrophilic polymer allows encapsulation of biomacromolecules for controlled release. As shown in Table 1, DX was modified with various functional groups, such as vinyl sulfone groups (—VS), or subsequently with Dithiothreitol (DTT) to create free thiol groups (—SH), carbazate or oxidized into aldehydes. The degree of modifications (DMs) of DX could be quantified with 1H-NMR spectroscopy and colorimetric methods, e.g., Ellman's assay. DM could be controlled with different reaction conditions [8], [9]. Briefly, the DM of VS could be adjusted with reaction time and the oxidation degree by the amount of NaIO₄.

TABLE 1
Hydrogel microparticles (HMP) formulation. Degree modification (DM) of precursor A, DM_{A} is larger than DM_{B} to
give excessive linkers for the peptide immobilization.VS: vinyl sulfone, SH: thiol, Cys: cysteine, L: lysine.

| | 1 | 2 |
|---|---|---|
| Precursor A (DM_{A}) | 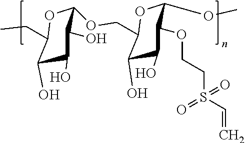 | 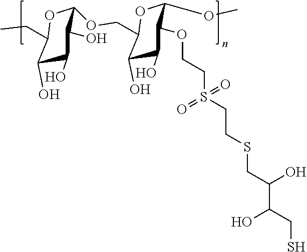 |
| Precursor B (DM_{B}; DM_{B} < DM_{A}) | 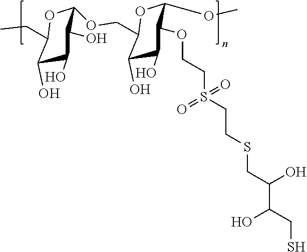 | 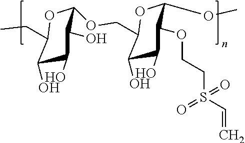 |
| Linker type on beads | 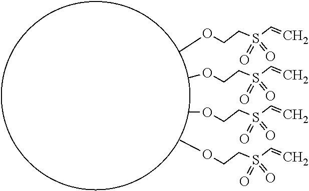 | 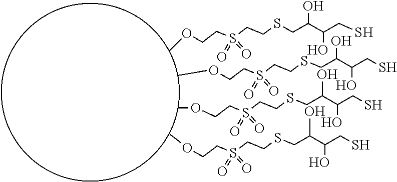 |
| Peptide | Thiol Michael addition between VS and SH on | Thiol Michael addition between SH and maleimide |
| conjugation | peptide with Cys-end | functionalized peptide |
| chemistry | | |
| | | 3 |
| | Precursor A (DM_{A}) | 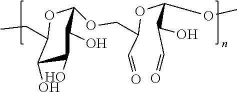 |
| | Precursor B (DM_{B}; DM_{B} < DM_{A}) | 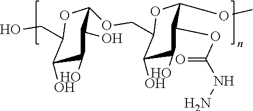 |
| | Linker type on beads | 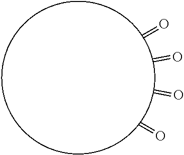 |
| | Peptide | Condensation between aldehyde and |
| | conjugation | primary amine on peptide N- |
| | chemistry | terminus (and side reaction on |
| | | peptide containing Lys) |

Subsequently, same concentrations of precursors A (DM_{A}) and B (DM_{B}, DM_{B}<DM_{A}) were mixed in volume ratio 1:1, to crosslink to form hydrogel polymer network. DM_{B} of limiting hydrogel precursor (i.e., precursor B in Table 1) would determine the crosslinking density of the resulting hydrogel, thus the mechanical stiffness. Whereas the unreacted functional groups of precursor A would be the linkers for the subsequent peptide conjugation. Therefore, the peptide density on HMIPs was approximately DM_{A}-DM_{B}. To increase surface peptide density, we could simply increase DM_{A}, fixing DM_{B}. To independently adjust HMPs stiffness, we could change polymer concentration of both precursors A and B, while fixing their DMs.

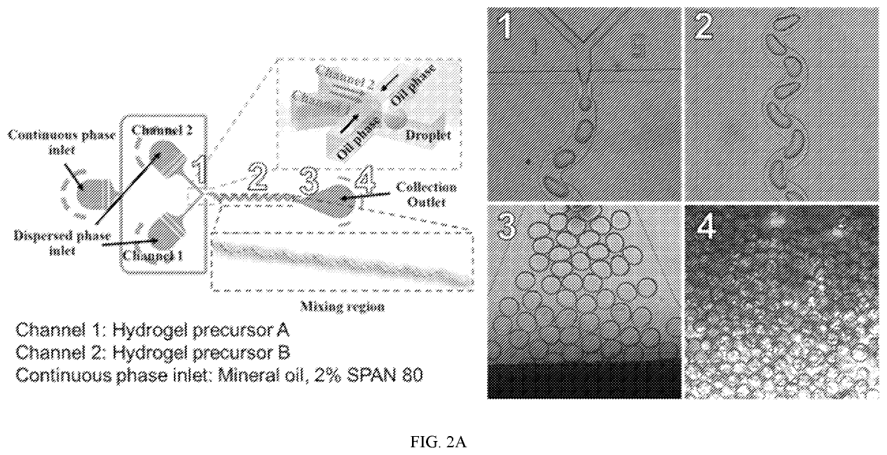

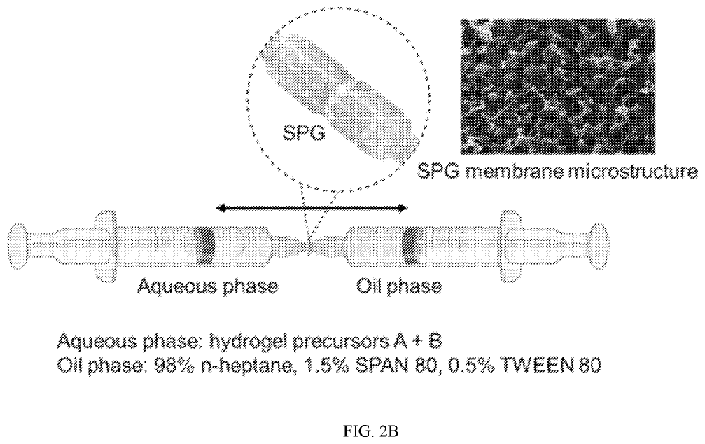

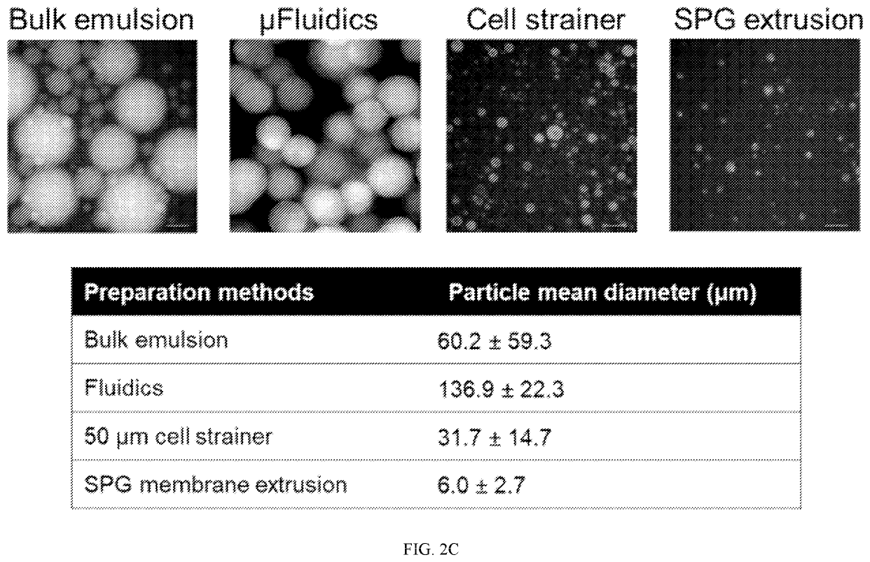

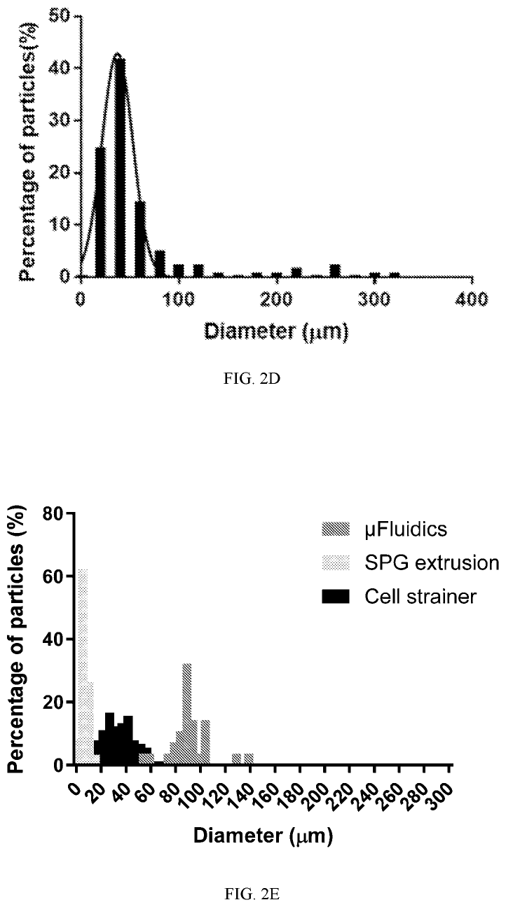

HMPs were formed in water-in-oil emulsions with different preparation methods to control the size and distribution, as shown in FIGS. 2A-2E. For bulk emulsion, cell straining, and SPG extrusion methods, the continuous phase (oil phase) consisted of 98% n-heptane, 1.5% Span 80 and 0.5% Tween 80; whereas the discontinuous phase (aqueous phase) was hydrogel precursors prepared in buffer with controlled pH. Conversely, microfluidics assisted droplet synthesis of HMPs was as previously reported [10]. The continuous phase contained 98% mineral oil and 2% Span 80. FIGS. 2A-2E shows that HMPs prepared in microfluidics have much lower PDI than bulk emulsion method. HMPs prepared in the former method had diameter of 136.9±22.3 μm (labelled as 100 μm for simplification). The HMIPs mixture from bulk emulsion was strained with 50 μm cell strainer to isolate the smaller particles, measured at 31.7±14.7 μm (40 μm). Conversely, HMPs prepared with SPG extrusion method were measured in average 6.0±2.7 μm (10 μm).

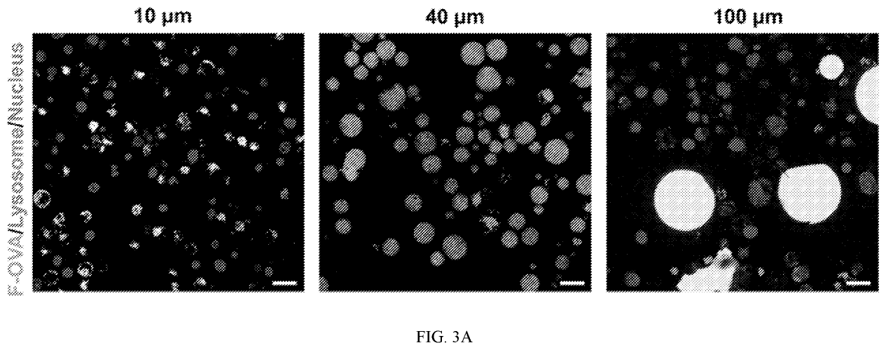

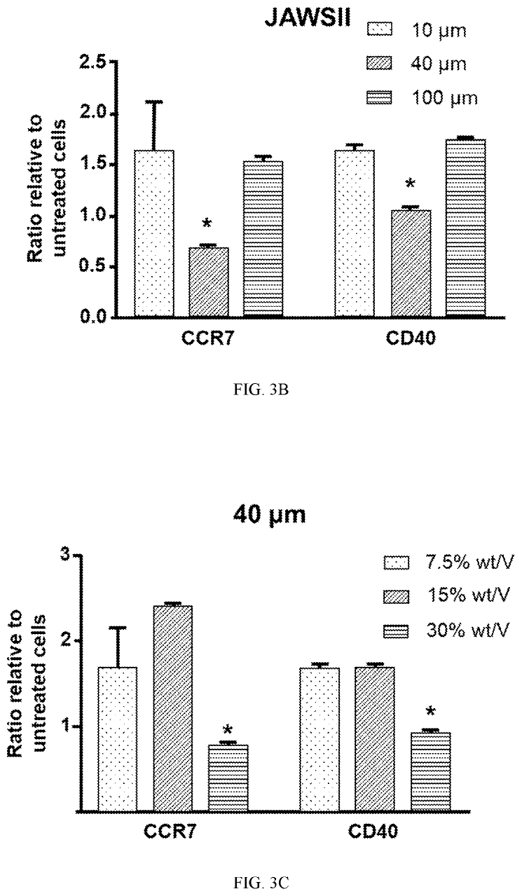

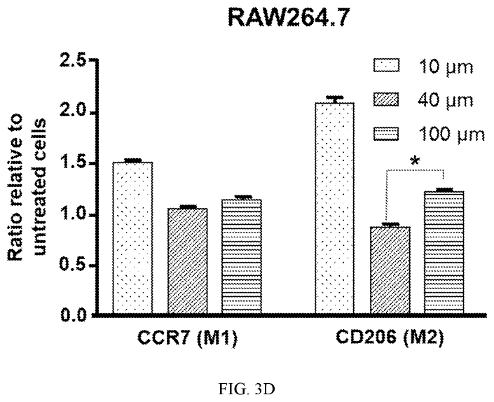

The HMPs were co-cultured with JAWSII, an immature dendritic cell line to investigate the effect of HMPs size and mechanical stiffness on cell-HMP interactions. The interaction was probed with FITC-probed polymers during HMP synthesis (green) and the cells were stained with Lysotracker to visualize lysosomes and late endosomal compartments (red), which were present during HMPs phagocytosis. For this experiment, DM_{A}=DM_{B} was selected, so that no reactive VS/SH/aldehyde group was present on HMP surface that would incur toxicity and influence the cell response. In our screening system design, it was desired to have HMP peptide support that induce less immune activation. As shown in FIG. 3A, HMPs (30% wt/V) of 40 μm size range appeared to induce less cell-gel interactions with lower induction of lysosome signals. Conversely, HMPs at 10 μm were efficiently phagocytosed by JAWSII on day 3 post-treatment, showing greater co-localization (yellow) of HMPs with the lysosomes. Whereas 100 μm HMPs were surrounded by activated cells. FIG. 3B also demonstrates the activation phenotypes of JAWSII, CCR7 (a lymph node homing marker) and CD40 (a co-stimulatory marker). Consistently, JAWSII co-cultured with 40 μm showed lowest activation of both markers, 0.7 for CCR7 and 1.05-fold for CD40 relative to untreated cells.

The mechanical strength of HMPs could adjusted with the concentrations of hydrogel precursors during preparations, while fixing the polymer molecular weights and DM. The stiffness increases with polymer concentrations. As shown in FIG. 3C, cells cultured on stiffest HMPs (30% wt/V) showed lowest activation in contrast to HMPs with 7.5% wt/V and 15% wt/V polymer concentrations. Furthermore, we also investigated the effect of HMP size on the response of macrophage line, RAW264.7. Similarly, HMPs of 40 μm neither induce M1 nor M2 polarization. DX-based HMP at 40 μm and 30% wt/V polymer concentrations appeared to be a bystander to dendritic cell and macrophage response in vitro; therefore, these physical parameters could be used to construct the microbeads for subsequent peptide immobilization and screening.

### Peptide Selection and Synthesis; Control of Combinatorial Peptide Display

The peptides which have been synthesized for screening are summarized in Table 2 and Table 3. Briefly, the peptide species could be categorized into IDR peptide, dipeptide, and phosphocholine-amino acid. The selection, design rationale and synthesis scheme will be described in the following sections. An adapter had been chemically added at the N-terminus or side chain of the peptide/amino acid candidates for coupling on HMPs.

TABLE 2
Peptide candidates with N-terminus modified with maleimide
hexanoate (m), cysteine (Cys) or
3-[(3-acrylamidepropyl)dimethylammonio]propanoate (ac)
(A3279; TCI chemicals, Tokyo, Japan),.

| | | N-terminus | Peptide | C- | MW | |
|---|---|---|---|---|---|---|
| # | peptide | adaptor | sequence | terminus | (Da) | pI |
| 1 | mLL-37 | Maleimide | FKRIVQRI | Amide | 1912.3 | 12.8 |
| | | hexanoate | KDFLR | | | |
| 2 | mIDR-1002 | Maleimide | VQRWLIVW | Amide | 1845.2 | 14 |
| | | hexanoate | RIRK | | | |
| 3 | mQ | Maleimide | QHREDGS | Amide | 1020 | 7.9 |
| | | hexanoate | | | | |
| 4 | mTLR2-1 | Maleimide | CSKKKK | Amide | 914.1 | 11.1 |
| | | hexanoate | | | | |
| 5 | C-SKKKK | Cys | SKKKK | Amide | 720.9 | 11.1 |
| 6 | C- | Cys | GGCSKKKK | Amide | 938.2 | 10.6 |
| | GGCSKKKK | | | | | |
| 7 | C-QHREDGS | Cys | QHREDGS | Amide | 930.9 | 7.9 |
| 8 | C-RGPPP | Cys | RGPPP | Amide | 625.7 | 14 |
| 9 | ac-GG | A3279 | GG | Carboxyl | 342.9 | 7.91 |
| 10 | ac-GA | A3279 | GA | Carboxyl | 357 | 7.04 |
| 11 | ac-GV | A3279 | GV | Carboxyl | 385.1 | 8.07 |
| 12 | ac-GL | A3279 | GL | Carboxyl | 399.1 | 8.75 |
| 13 | ac-GF | A3279 | GF | Carboxyl | 419.1 | 7.81 |
| 14 | ac-GC | A3279 | GC | Carboxyl | 389.1 | 6.65 |
| 15 | ac-GD | A3279 | GD | Carboxyl | 387 | 3.24 |
| 16 | ac-GK | A3279 | GK | Carboxyl | 413.9 | 11.47 |

MW: molecular weight,
pI: isoelectric point.

TABLE 3
Peptide candidates with side chain hydroxyl group modified
with zwitterionic phosphoryl choline moiety.

| # | Species | Side chain adaptor | Amino acid sequence | C-terminus | MW(Da) | pI |
|---|---|---|---|---|---|---|
| 17 | ncp-S | ncp | S | Carboxyl | 367.36 | 11.6 |
| 18 | ncp-T | ncp | T | Carboxyl | 381.39 | 11.63 |
| 19 | ncp-Y | ncp | Y | Carboxyl | 443.46 | 11.49 |

MW: molecular weight, pI: isoelectric point.

### IDR Peptide Synthesis and Display (no. 1-8)

The immunomodulatory properties of IDR peptides have been reported by various studies. LL-37, a human AMP exhibited chemotactic and modulatory activities on innate immune cells, particularly dendritic cells. It has been used for promoting wound healing and inhibiting for formation of biofilms [11]. In addition, the intranasal immunization of antigens with LL-37 as adjuvant elicited potent antigen-specific humoral responses mediated by systemic IgG and mucosal IgA, as well as cytotoxic T-lymphocyte stimulation [12], [13]. A list of LL-37 derived peptides and analogues with therapeutic functions have been identified and summarized in this review [14]. In this study, we selected FKRIVQRIKDFLR (SEQ ID NO: 1) (or FK16), which was one of the shortest peptide derivatives of LL-37, and the antimicrobial and anti-cancer activity of FK16 have been demonstrated in other study [29].

IDR-1002 (VQRWLIVWRIRK; SEQ ID NO: 2) is a synthetic cationic peptide (pI>14) which was shown to inhibit bacterial infections via chemokine induction on human PBMCs and enhanced leukocyte recruitment. In fact, IDR-1002 was selected from a library of bactenecin synthetic derivatives with greatest potency in inducing chemokines on modulation activities on innate immune cells [16]. Conversely, Q-peptide (QHREDGS; SEQ ID NO: 3) exhibited both pro-inflammatory and anti-inflammatory responses on macrophages in vitro. Moreover, the coating of Q-peptide on chitosan-based hydrogel also showed excellent wound healing responses in diabetic mouse model and tissue repair in myocardial infarction model [17], [18]. The peptide is derived from angiopoietin-1, a ligand to integrin which could help to maintain barrier functions in endothelial cell walls, promoting blood vessel health and remodeling [19]-[21].

On the other hand, CSKKKK (SEQ ID NO: 4) is derived from Pam3CSK4. This synthetic lipopeptide is a potent TLR2 agonist, of which the peptide head modulates the extent of ligand-receptor interactions [22]. Here we added a C₆ or Gly-Gly linker between the HMPs and peptide N-terminus to investigate the effect of flexibility of receptor-ligand interactions on the cell response. RGPPP (SEQ ID NO: 7) was isolated from protein lysate of a plant Pseudostellaria heterophylla, which has been used in traditional Chinese medicine. The herb extract demonstrated immune stimulatory activities and anti-tumor responses. Particularly, the RGPPP (SEQ ID NO: 7) peptide exhibited potent immune stimulating responses, in inducing macrophages to produce TNF-α and upregulation of TLR2 expression.

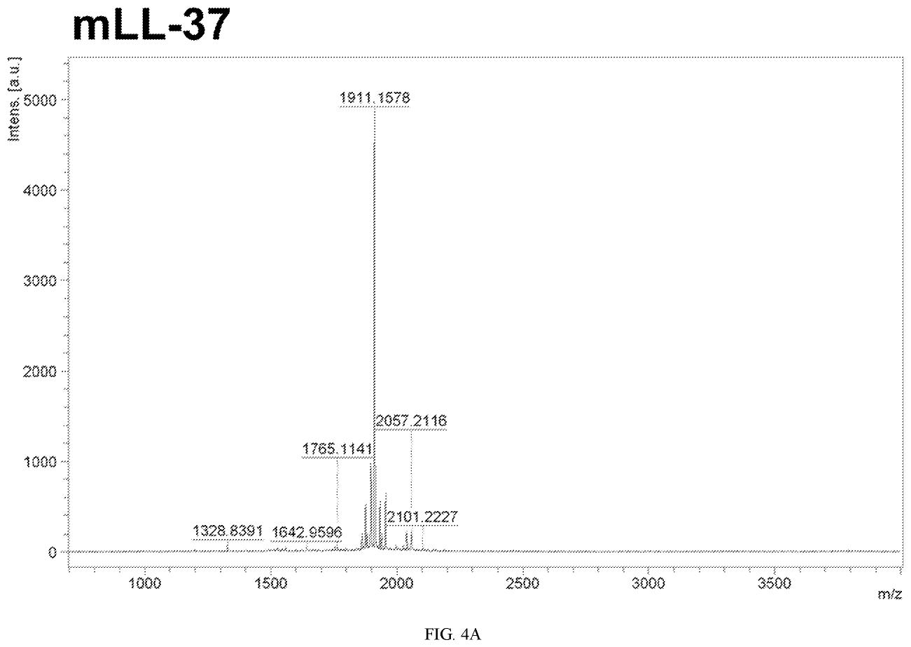

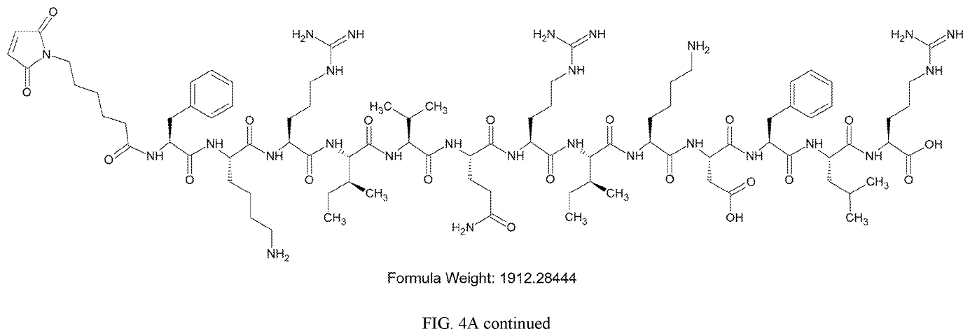

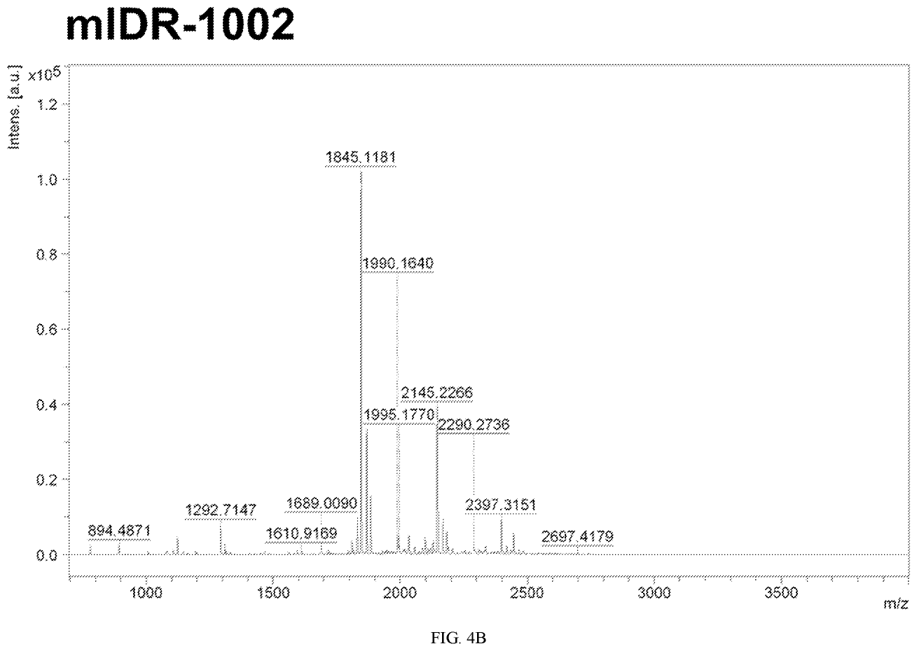

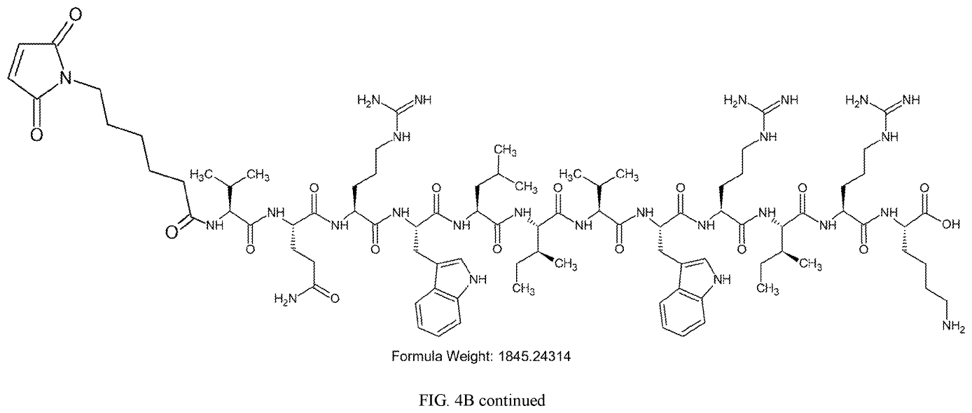

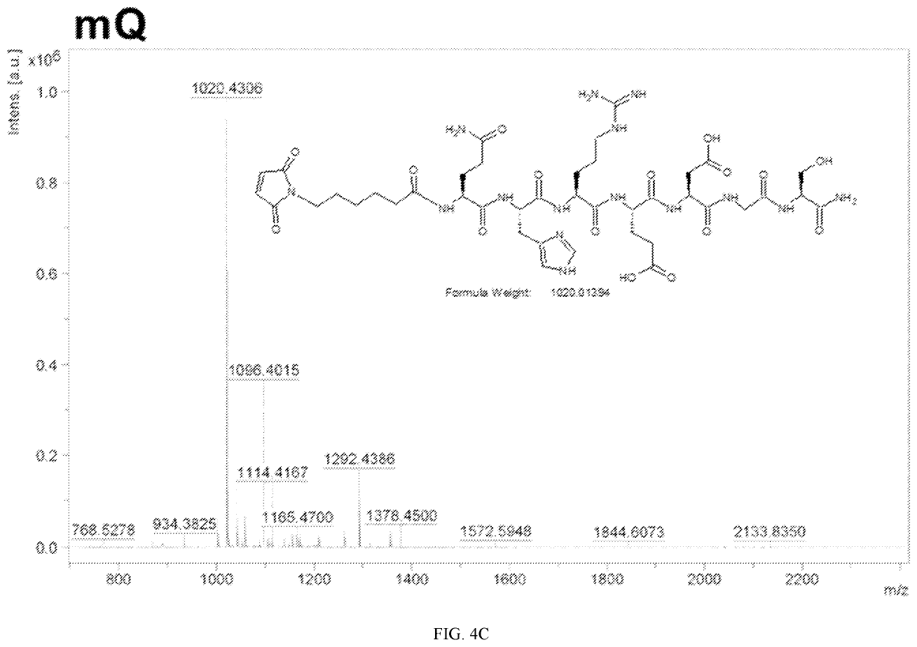

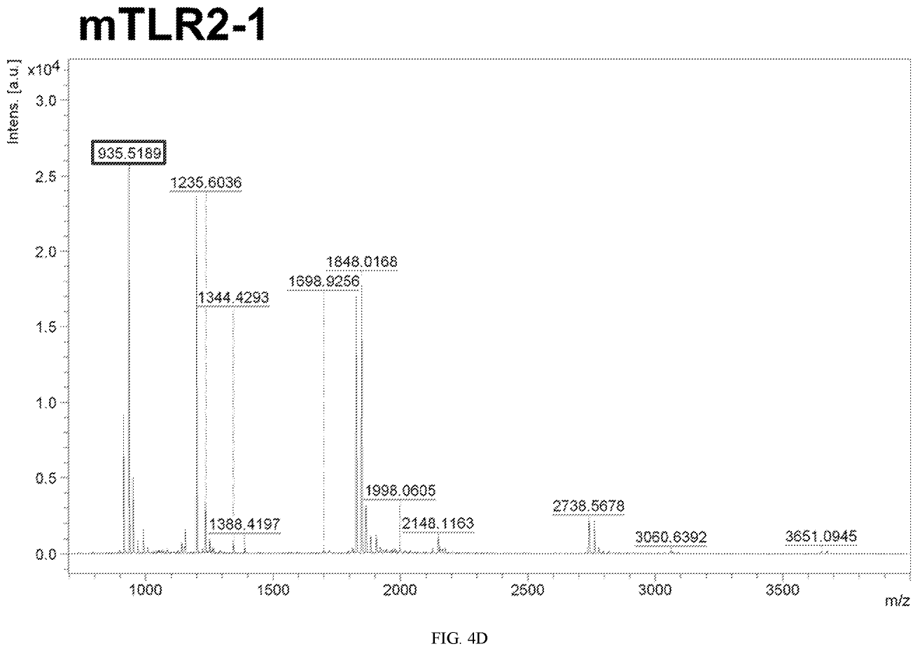

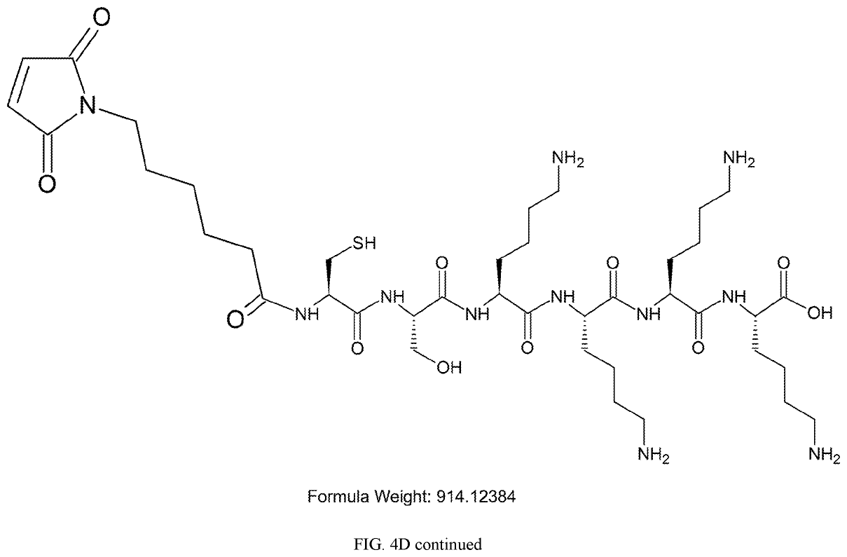

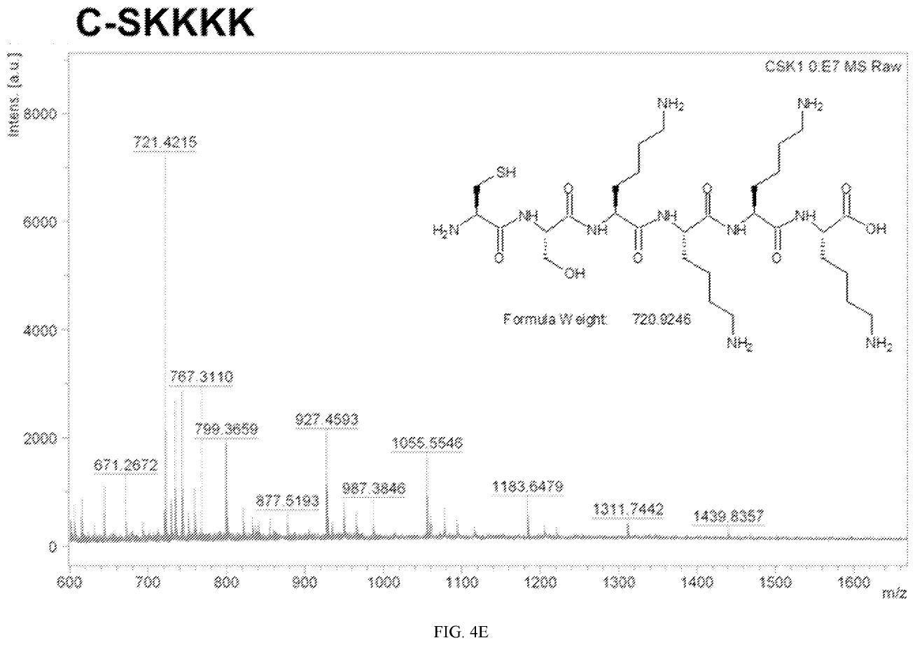

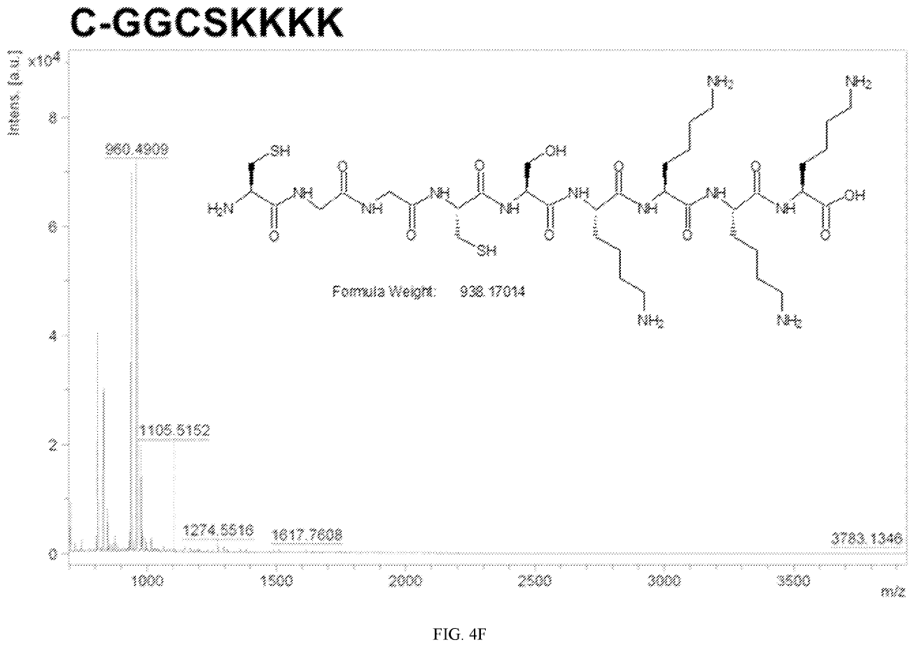

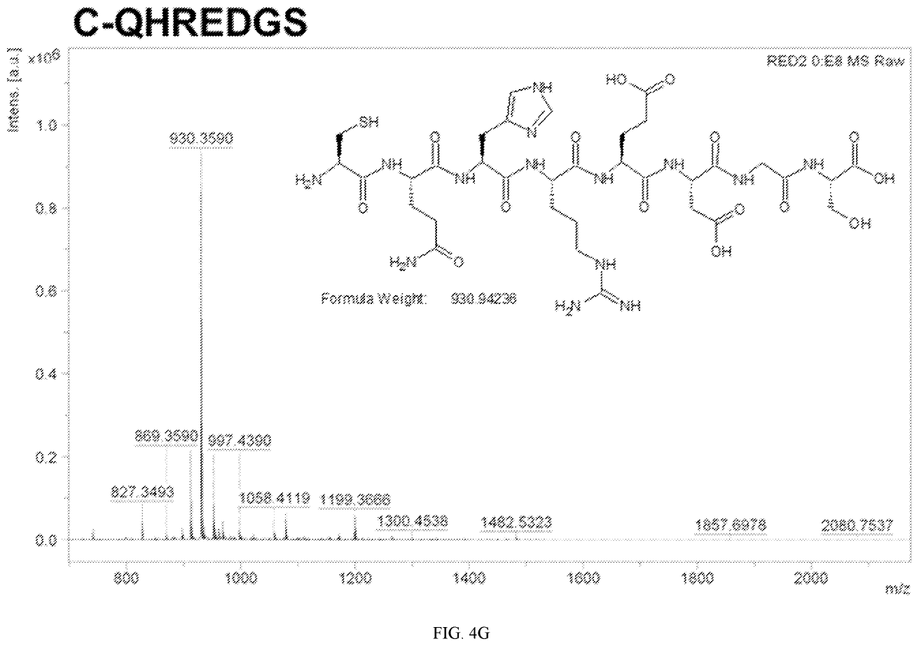

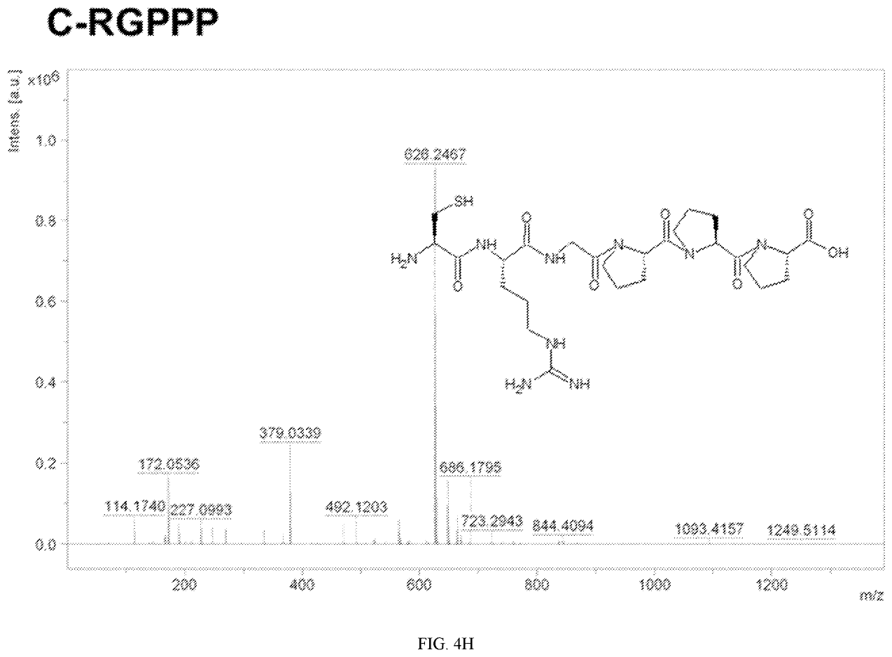

In this study, peptide species no. 1 to 8 were produced via automated microwave-facilitated solid phase peptide synthesis (SPPS). Rink amide resins were used and HBTU/HOBt/DIPEA as catalyst. In peptide species no. 1 to 4, maleimide hexanoate (m) was added to the Fmoc-deprotected peptide via condensation between primary amine and carboxylate during SPPS. Maleimide-end peptide would be coupled with HMPs with free thiols via efficient Michael addition. Conversely, in peptide species 9 to 6, cysteine residue (Cys) was added to the N-terminus of peptide candidate during solid phase synthesis to give free thiols and amine for coupling on HMPs with free VS or aldehyde respectively. The final products were cleaved from the resins with TFA/EDT (95/5) and precipitated in cold ether. The MALDI-TOF of the peptide species is shown in FIGS. 4A-411.

### Dipeptide Synthesis and Display (No. 9-16)

Muramyl dipeptides (MDPs) are derived from bacterial peptidoglycan cell wall. They are the smallest structural component that still retain immunostimulatory activity. MDPs consist of a sugar unit linked to two amino acids. A typical MDP is made of N-acetylmuramic acid linked by its lactic acid moiety to the N-terminus of L-alanine and D-isoglutamine dipeptide. MDPs are ligands to TLR2 and NOD-like receptors (NR)[23]. They have been used as adjuvant in several vaccine formulations for mucosal immunizations in the past [24]. Several dipeptide variations have been introduced to improve the adjuvant activity. Particularly, studies demonstrated that replacing L-alanine with L-serine or D-isoglutamine with D-glutamic acid demonstrated higher immune stimulatory activity [25].

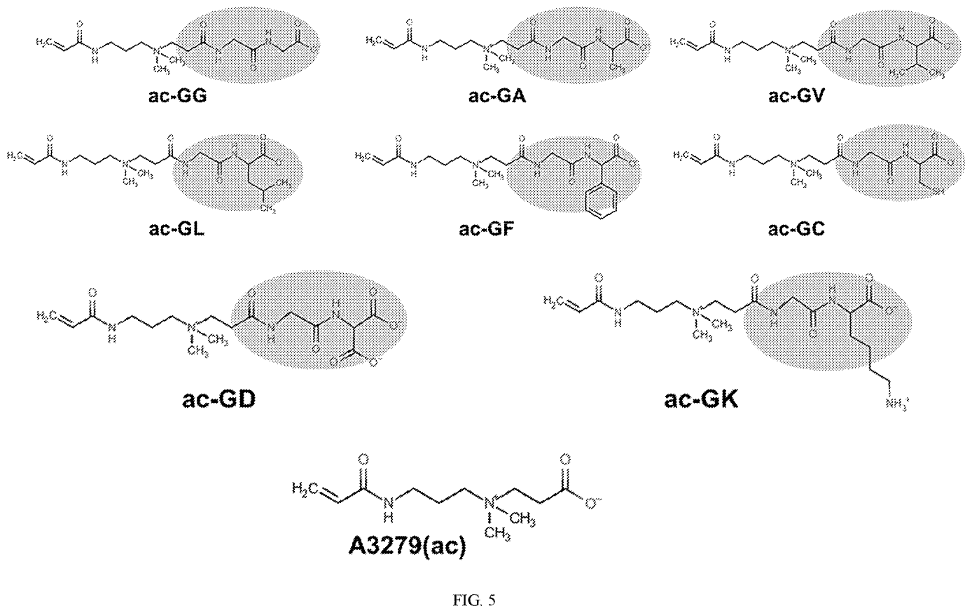

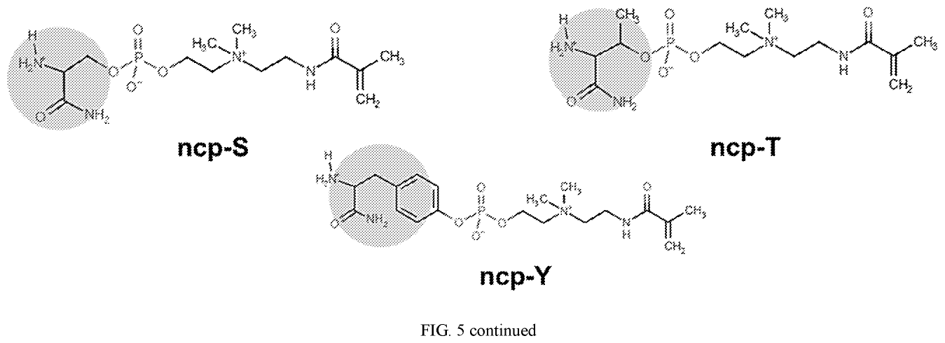

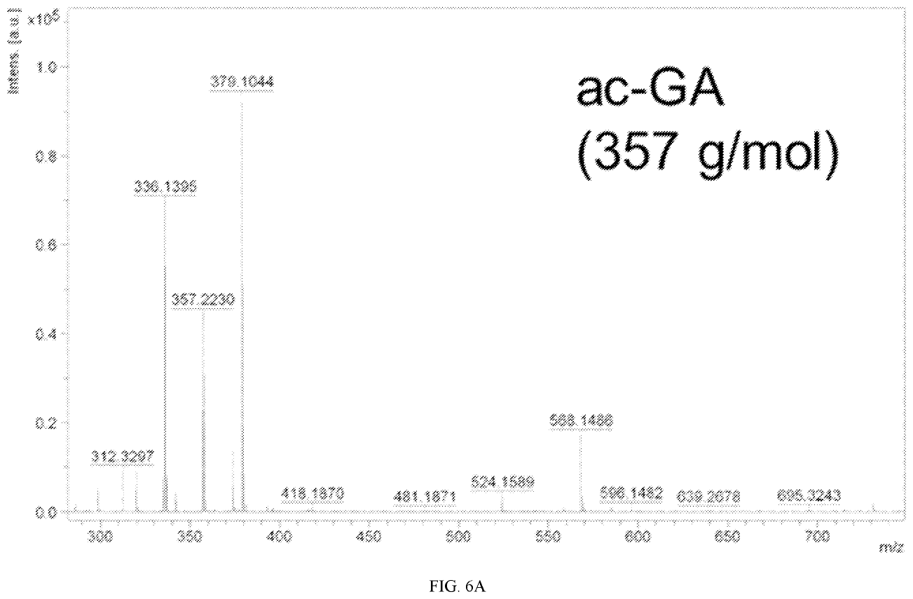

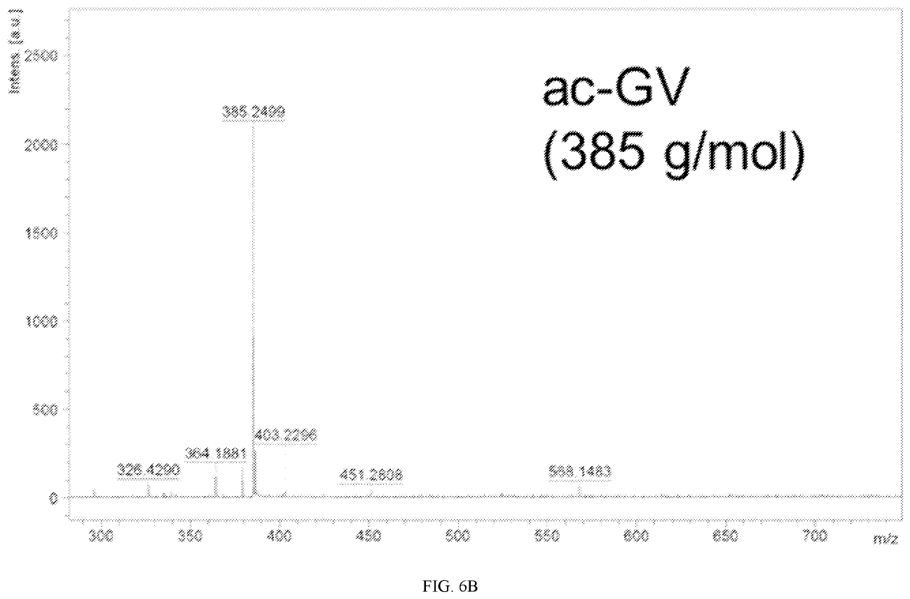

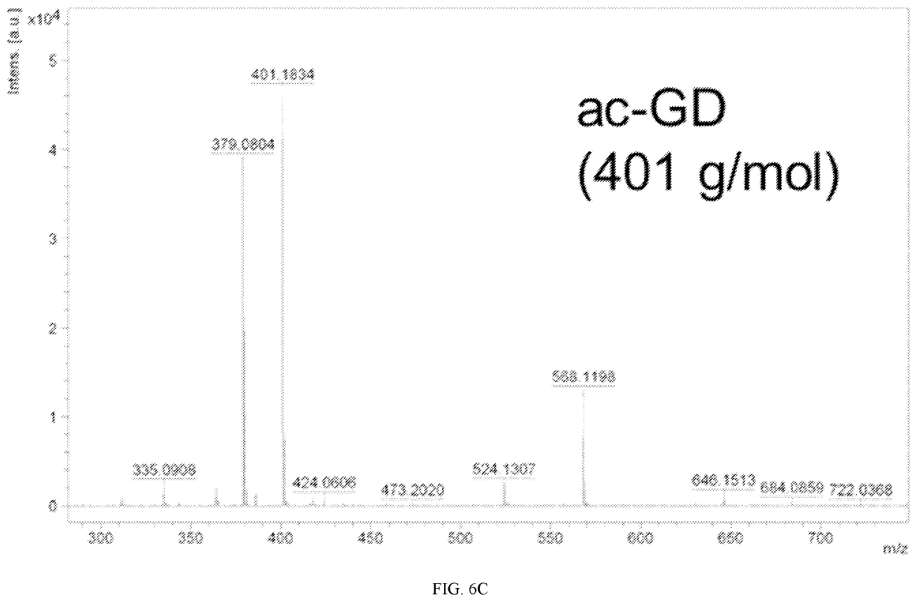

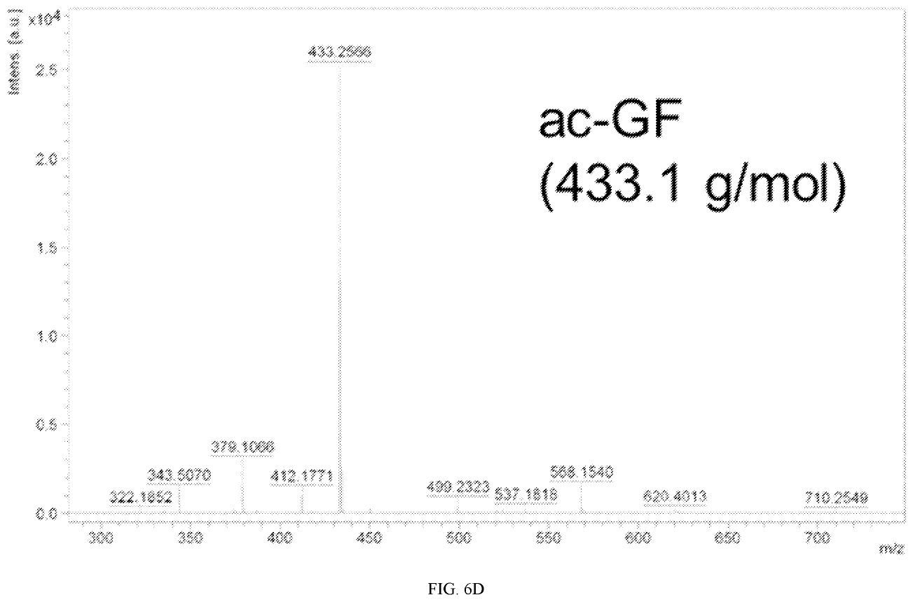

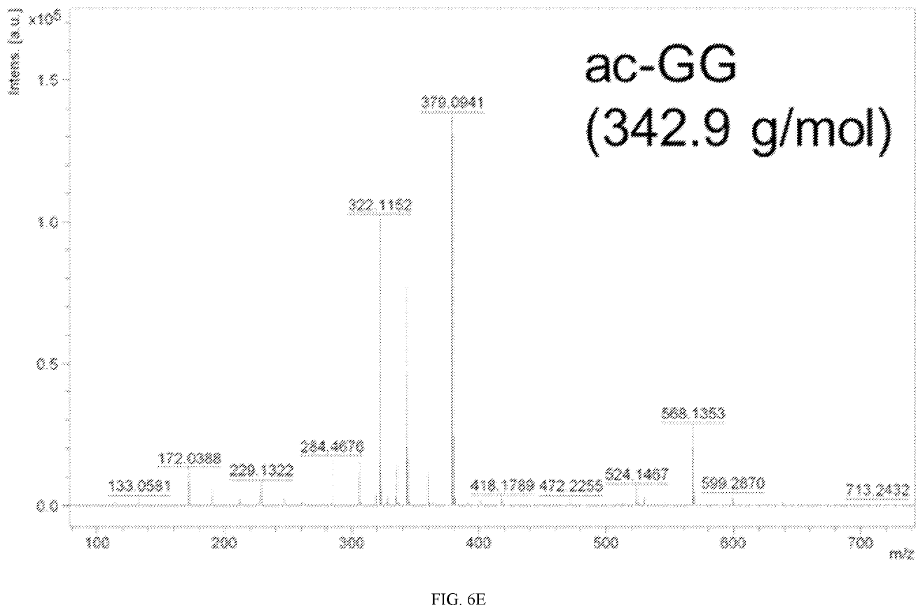

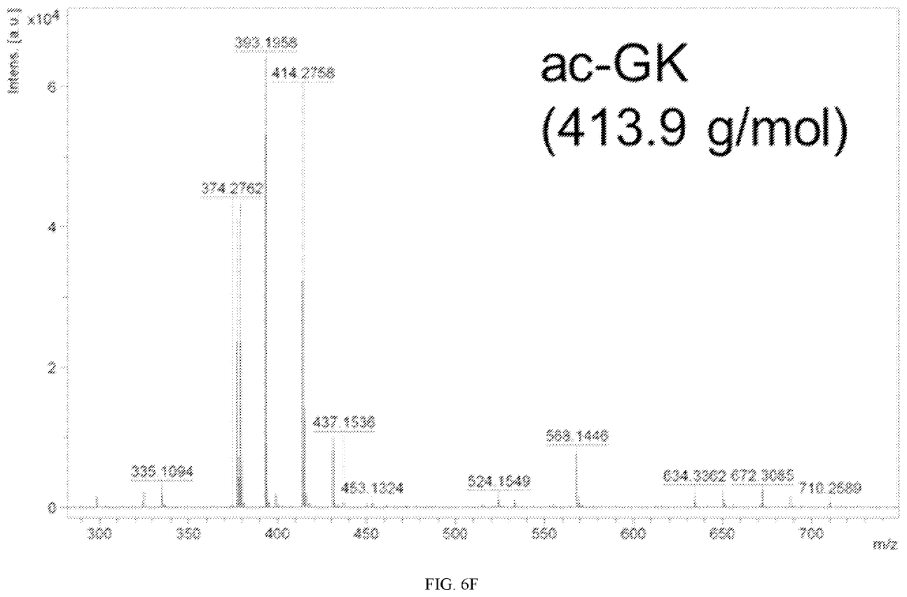

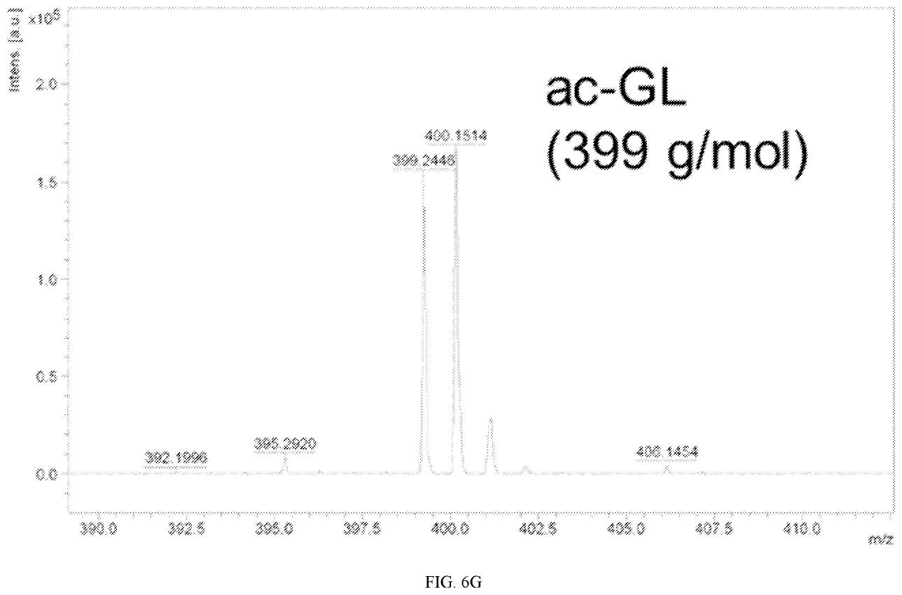

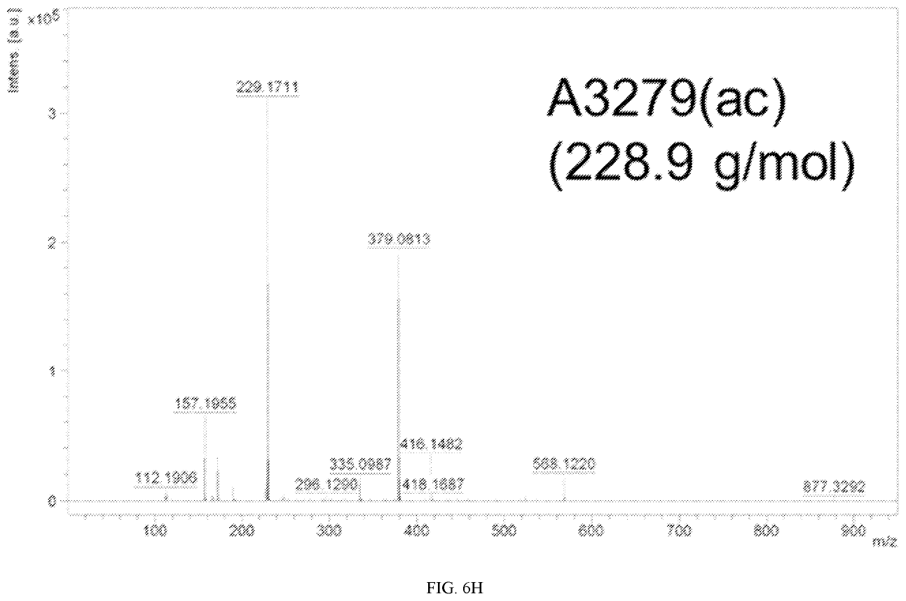

Herein, we aim to utilize the HMP-based screening platform to screen for the immunomodulatory activity of different combinations of dipeptide. A library of dipeptides was synthesized via SPPS (parallel synthesis), with reactions summarized in Scheme 1. The general structure of dipeptide species of generation 1 library was aG-X, as shown in Table 1. In our structural design, a cationic adapter, 3-[(3-acrylamidepropyl)dimethylammonio]propanoate (ac) (a) was added at the N-terminus of dipeptide via condensation between carboxylate and primary amine group. The quaternary amine group of ac serves to improve cell-gel interactions and provide flexibility on ligand-receptor interactions, whereas the acrylamide group is for coupling on HMPs via thiol-acrylamide Michael addition. The N-terminus amino acid was fixed with Gly (as simple analogue to Ala), while the C-terminus amino acid was selected from Gly, Ala, Val, Leu, Phe, Cys, Asp or Lys (no. 9 to 16). The chemical structures of all aGX species are depicted in FIG. 5. The MALDI-TOF of the dipeptide are in FIGS. 6A-6H.

### Scheme 1: Synthesis Route of Peptide Species 9 to 16.

The coupling of first amino acid variable (AA) to Fmoc-deprotected Wang-resin was performed on automated microwave assisted SPPS (4 eq. protected AA, 4 eq. 0.6 M HBTU, 0.5 M HOBt, 8 eq. 2 M DIPEA in DMF). 1. Gly (G) was coupled to Fmoc-deprotected AA in parallel synthesis in separate vials using 4 eq. (Fmoc)-Gly-OH, 4 eq. 0.6 M HBTU, 0.5 M HOBt, 8 eq. 2 M DIPEA in DMF, RT overnight. 2. Fmoc deprotection with 20% V/V piperidine in DMF for 2 hours, RT. Resins were washed extensively in DMF (4 times) and DCM (4 times). 3. 4 eq. A3279 (3-[(3-acrylamidepropyl)dimethylammonio]propanoate (ac)), 4 eq. DCC/HOBt in DMF, overnight at RT. Resins were washed with DMF, DCM and methanol. 4. The final product was decoupled with TFA/TIPS/H₂O (95/2.5/2.5) for 2 hours, RT and precipitated in cold ether/DCM.

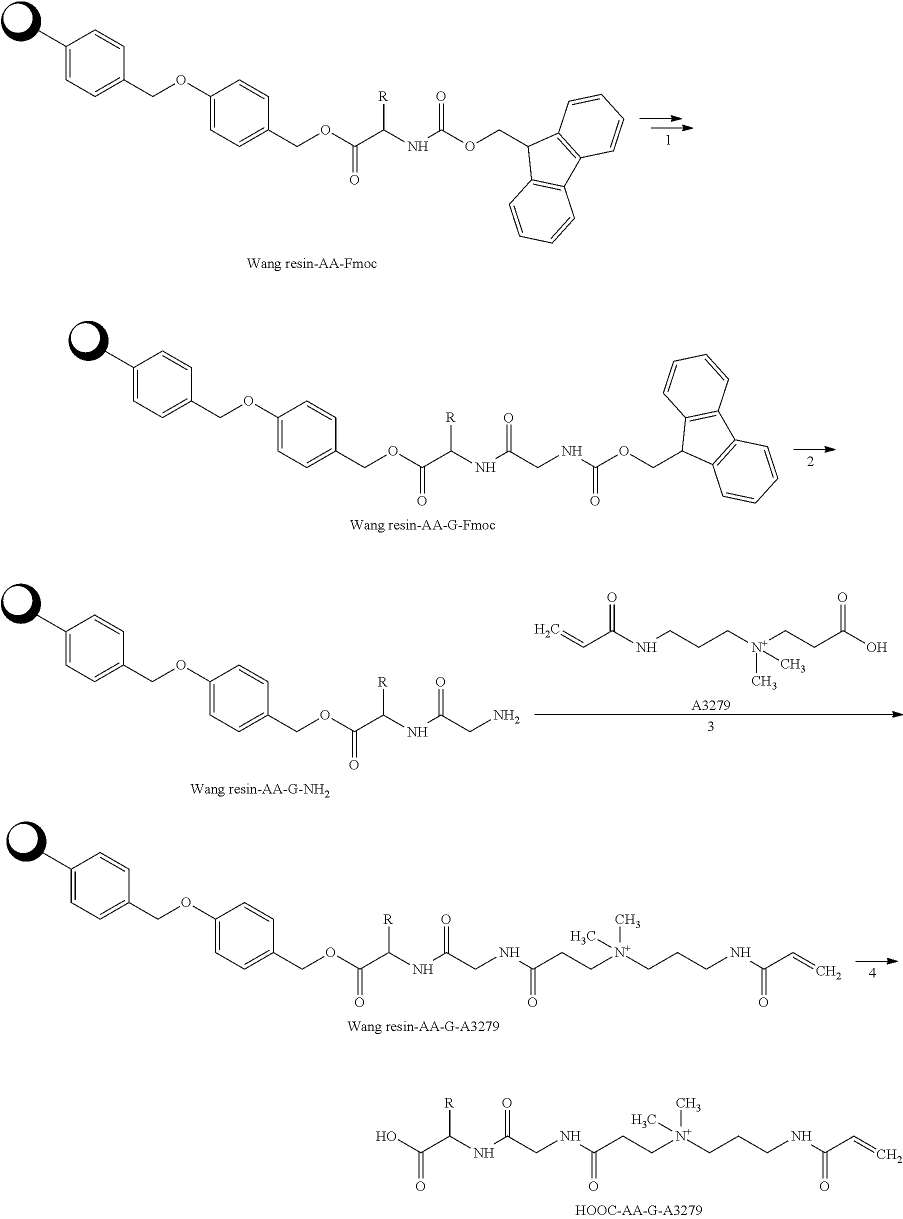

### Scheme 2: Synthesis Route of Peptide Species 17 to 19.1.

The coupling of first amino acid variable (AA) to Rink Amide resin was performed on automated microwave assisted SPPS (4 eq. protected AA, 4 eq. 0.6 M HBTU, 0.5 M HOBt, 8 eq. 2 M DIPEA in DMF). 2. ter-Butyl deprotection with H₃PO₄ (85% aq) in DMF for 12 hours 5 at RT. Wash resins with excessive DMF. 3. 4 eq. 2-chloro-1,3,2-dioxaphospholane 2-oxide and 0.5 eq. triethylamine were diluted in ice-cold DMF and added dropwise to the resin reaction mixture, 30 min on ice and stirred at RT for 3 hours. 4. 4 eq. of N-[3-(Dimethylamino)propyl]methacrylamide was dissolved in acetonitrile and added into reaction mixture, stirred at 55° C. in silicone oil bath, overnight. The resins were cleaned up with DMF, DCM then methanol. 5. The final products were cleaved with TFA/TIPS/H₂O (95/2.5/2.5) and precipitated with cold ether/DCM.

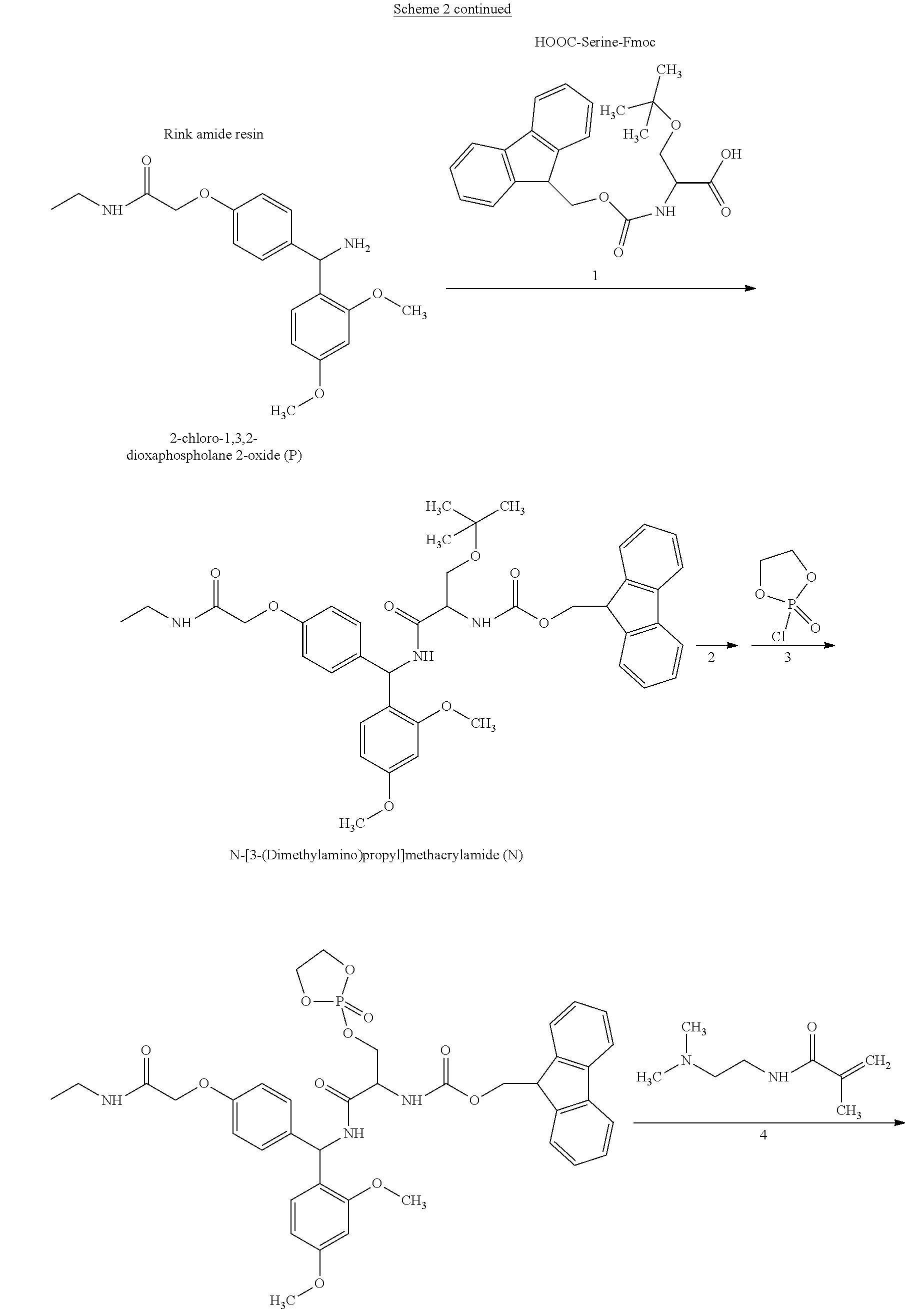 [SMILES: CCNC(=O)COc1ccc(C(N)c2ccc(OC)cc2OC)cc1 ; CC(C)(C)OCC(NC(=O)OCC1c2ccccc2-c2ccccc21)C(=O)O ; CCNC(=O)COc1ccc(C(NC(=O)C(COC(C)(C)C)NC(=O)OCC2c3ccccc3-c3ccccc32)c2ccc(OC)cc2OC)cc1 ; O=P1(Cl)OCCO1 ; CCNC(=O)COc1ccc(C(NC(=O)C(COP2(=O)OCCO2)NC(=O)OCC2c3ccccc3-c3ccccc32)c2ccc(OC)cc2OC)cc1 ; C=C(C)C(=O)NCCN(C)C]

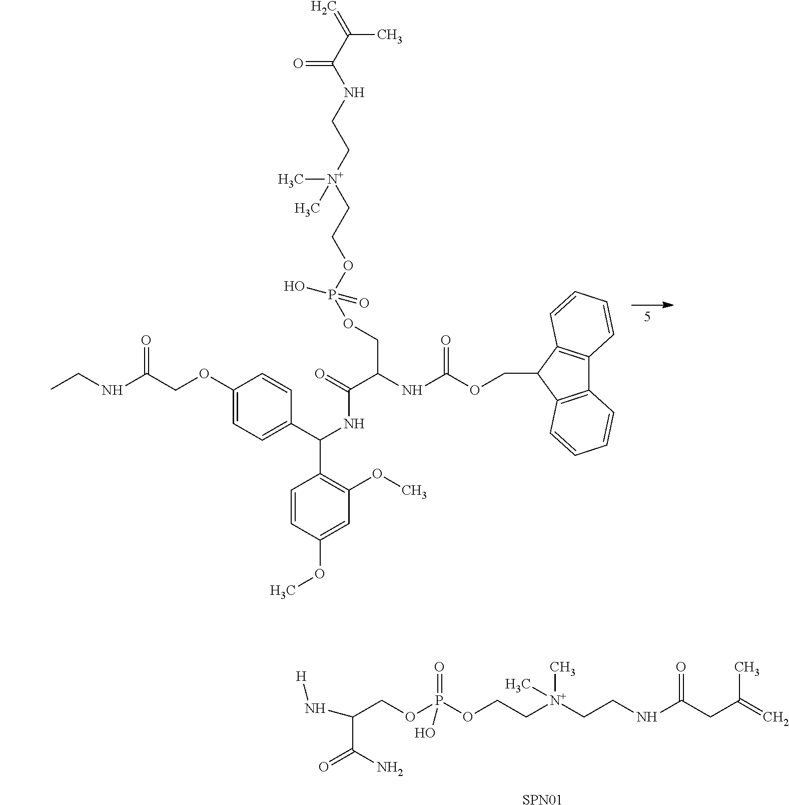 [SMILES: C=C(C)C(=O)NCC[N+](C)(C)CCOP(=O)(O)OCC(NC(=O)OCC1c2ccccc2-c2ccccc21)C(=O)NC(c1ccc(OCC(=O)NCC)cc1)c1ccc(OC)cc1OC ; C=C(C)CC(=O)NCC[N+](C)(C)CCOP(=O)(O)OCC(N)C(N)=O ; "SPN01"]

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1 De Novo Synthesis Scheme of Phosphocholine-Amino Acid (NPC-X, No. 17-19)

Lipoamino acids are generally found in bacteria and algae. The general structure is made of hydrophobic hydrocarbon tail(s) and a hydrophilic amino acid moiety, usually Ser, Lys, Gly or Orn [26]. A library of synthetic lipoamino acid analogues have been synthesized and used as nucleic acid delivery vehicles for targeted gene silencing in liver parenchymal cell in vivo, while no severe systemic immune-related adverse events were reported [27].

Nonetheless, lipoamino acids were shown to induce differential immune responses in vitro and in vivo. The cationic lipoamino acids are potential adjuvants for antigen/epitope vaccination. The conjugation of lipo-oligo-Lys with multiple antigen peptides at C-terminus greatly enhanced the epitope-specific humoral responses, in contrast to the native peptides [28]. Conversely, the phosphatidylserine in mammalian cells was associated with anti-inflammatory responses. The natural lipid was presented on the plasma membrane during apoptosis, acting as a ‘eat me’ signal to promote phagocytic clearance of the dead cells by macrophages while maintaining immune tolerance to self-antigens. Furthermore, a study constructed zwitterionic phosphatidylserine de novo the in vitro results demonstrated that the coating of hydrogel with the zwitterionic entities could exert anti-fouling responses in suppressing protein and macrophage adhesion. Moreover, the zwitterionic lipoamino acid could suppress the immunogenicity of uricase more effectively than the unmodified lipoamino acid in vivo [29].

We built a zwitterionic phosphocholine-functionalized amino acid and screen for their immunomodulatory activity on the DX-based HMP support system. The reactions are summarized in Scheme 2. Briefly, hydroxyl-side chain containing amino acid, Ser, Tyr and Thr were coupled to Wang-resin in DMF. Then, the hydroxyl protective group, t-Bu, with phosphoric acid (~85% V/V) in DMF for 12 hour, at room temperature as previously reported. The resins were washed with DMF extensively, followed by the addition of triethylamine (0.5 eq.) and 2-chloro-1,3,2-dioxaphospholane 2-oxide (4 eq. to hydroxyl groups on amino acid) dropwise diluted in DMF. Reaction was maintained on ice bath for 30 min, then 3 hours at room temperature with stirring. The resins were washed with DMF extensively to remove excessive soluble reactants, and followed by the addition of N-[3-(Dimethylamino)propyl]methacrylamide (4 eq.) dissolved in acetonitrile. Reaction occurred in 55° C., silicone oil bath overnight. Finally, the resins were cleaned up with DMF, DCM, then methanol washing. The final products (npc-X; where X represents amino acid, Ser, Tyr or Thr) were cleaved with TFA/TIPS/H₂O (95/2.5/2.5) and precipitated with cold ether/DCM. The chemical structures of npc-X are shown in FIG. 5.

### Example 2 Compatibility with High Throughput Screening Assays (Cell-Based)

DC and macrophage reporter cell lines were adopted to screen for the NFκB signaling activity. NF-κB is a transcription factor that regulates a large array of genes involved in pro-inflammatory responses. Many PRRs, such as TLRs and CLRs are conserved activators of NF-κB signaling. Therefore, we can infer the peptide-receptor interactions (synergism, antagonism, additivity, or null response) by detecting and measuring NFκB activity with a reporter system. As shown in FIG. 2B, DX HMPs (40 μm, 30% wt/V) exhibited low level of immunogenic activation in JAWSII, which could be used to define the baseline for M1/M2 or immunogenic/tolerogenic induction of macrophage or JAWSII respectively. For instance, the immunogenic or M1 polarizing peptide species would induce higher NFκB activity than bare HMPs. Conversely, tolerogenic or M2 polarizing peptides would suppress the background NFκB activity. LPS, a potent TLR4 agonist and/or CpG (TLR9) would be used as positive control for M1/immunogenic induction, whereas IL-10 as positive control for M2/tolerogenic induction.

RAW Blue™ is a commercially available reporter cell line. In the presence of agonists of PRRs (except TLR5, NOD1), the endogenous production of NFκb and AP-1 could induce the reporter system to generate SEAP. SEAP levels were monitored with colorimetric assay using QUANTI-Blue™. On the other hand, DC reporter cell line was generated in-house, by transfecting JAWSII with NanoLuciferase® reporter system, pNL3.2.NF-κB-RE[NlucP/NF-κB-RE/Hygro] (Promega, US) according to manufacturer's instructions. Cells were cultured for 72 hours prior to treatment with dipeptide candidates (no. 9 to 16) at 10 ng/mL. The NFκB signaling activity could be reflected on the luminescence from the enzymatic activity of luciferase.

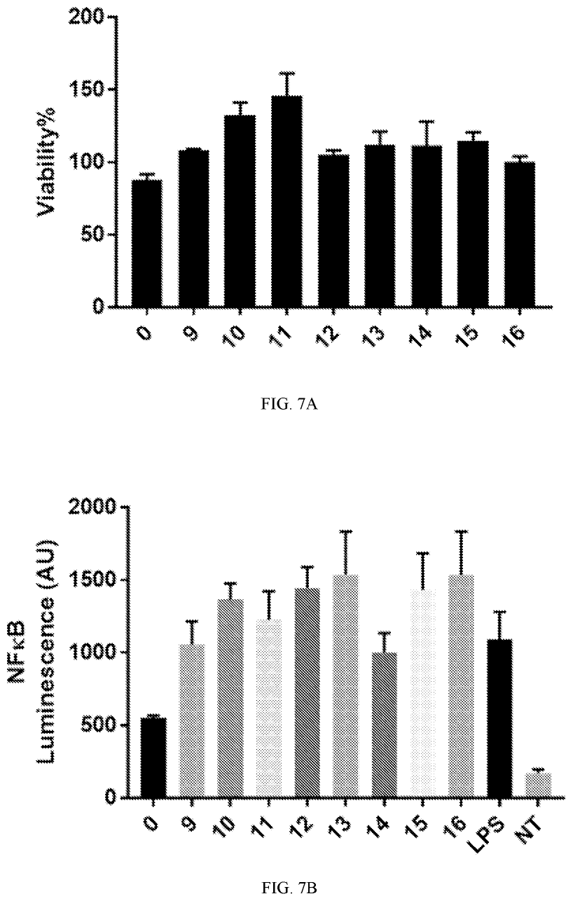

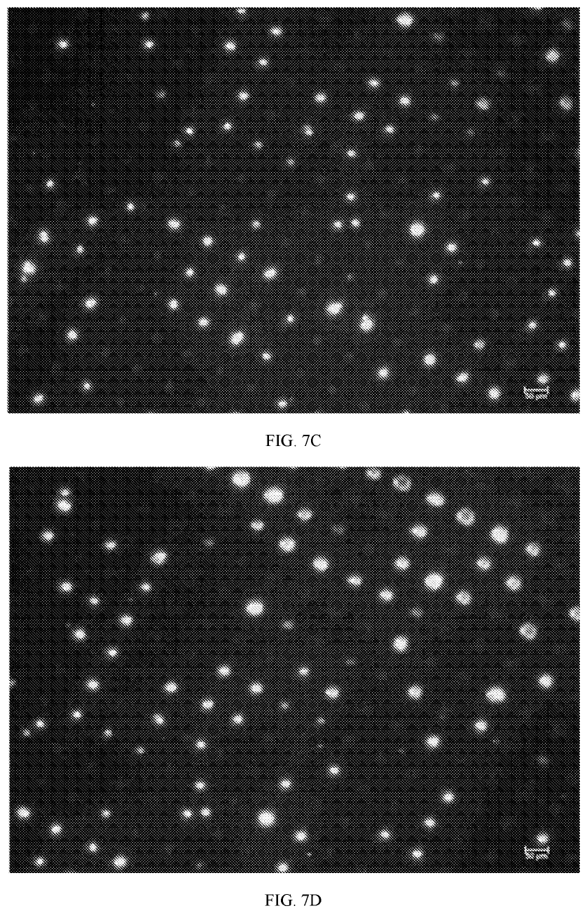

As shown in FIG. 7A, HMP-dipeptide conjugates exhibited good biocompatibility with >80% viability from MTT assay. In FIG. 7B, we also observed that the dipeptide species could significantly induce NFκB activity in JAWSII, as compared to blank HMPs (0) and non-treated cells (NT). Species 9 (ac-GG) and 14 (ac-GC) (10 ng/mL) could induce ~2-fold higher in NF-κB activity, which were comparable to 100 ng/mL LPS treatment as positive control. Conversely, the remaining dipeptide candidates could induce >2-fold increase in NF-κB. Particularly, dipeptide 13 (ac-GF) and 16 (ac-GK) showed highest response, implicating strong immunogenic adjuvant activity on JAWSII.

### Example 3—Compatibility with High Throughput Screening Workflows and Barcoding

Biomaterials and vaccine adjuvant development are both rapidly evolving fields. To keep pace with them, researchers have adapted high throughput screening (HTS) system to identify substrates and culture conditions that are appropriate to DC and macrophages maturation phenotypes. Libraries of materials and adjuvants can be assayed in vitro for their bioactivity, in our case, the propensity in inducing tolerogenic/M2 or immunogenic/M1 phenotypes. The major advantages of HTS are the acceleration in material bio-characterizations, guidance in material design and optimization in an unbiased fashion. Moreover, with HTS, we could expand our assay from single readout of NFκB activity to a vast assay of gene activity with sequencing and transcriptomics.

In our study design, the solid phase HMP-immobilized peptide candidates can be well-fitted in HTS assays. The workflow consists of 4 main modules: 1) HMP-peptide library barcoding (“catalogue indexing”), 2) associating barcode with cell response, i.e., gene products (mRNA) (“barcode generation and reading”), 3) pooling of barcoded gene products for multiplexed sequencing and 4) data analysis and interpretation.

In the proposed workflow, the beads are separately loaded in 96- or 384-well containing cells for parallel cell assays for various HMP-peptides. After the treatment, the cells were fixed for subsequent in-cell reverse transcription (RT), which has been optimized in other studies [29]. During in-cell RT, we barcode the mRNA products of well-specific cells with unique indexing primers, corresponding them to the given HMP-peptide perturbations. To achieve this, the unique indexing primers are loaded into corresponding HMP-peptide during HMP preparations and released with induced hydrogel degradation, as previously reported in other studies [30]. The cells are pooled and loaded into microwells (as shown in FIGS. 7C-7D). The microwells diameter and depth are optimized to control the cell loading, e.g., number of cells per well and number of wells with cells. Moreover, low speed centrifugation is applied to increase the cell loading efficiency. The barcoded (complementary DNA-labelled) microbead is then seeded one in each well to fish/call out the corresponding barcoded RT products upon cell lysis and DNA-DNA hybridization. The fished cDNA is then amplified by PCR. The PCR products are pooled and subjected to sequencing. After sequencing, the transcriptome of individual cells is assembled by combining the reads that contain the same barcode combination.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A hydrogel microparticle comprising at least one polysaccharide and at least one peptide, wherein the at least one peptide comprises an N-terminus adapter or a side chain adapter.
Embodiment 2. The hydrogel microparticle of embodiment 1, wherein the at least one polysaccharide is dextran.
Embodiment 3. The hydrogel microparticle of embodiment 1, wherein the at least one peptide comprises the amino acid sequence FKRIVQRIKDFLR (SEQ ID NO: 1), VQRWLIVWRIRK (SEQ ID NO: 2), QHREDGS (SEQ ID NO: 3), CSKKKK (SEQ ID NO: 4), SKKKK (SEQ ID NO: 5), GGCSKKKK (SEQ ID NO: 6), RGPPP (SEQ ID NO: 7), GG, GA, GV, GL, GF, GC, GD, GK, S, T, or Y.
Embodiment 4. The hydrogel microparticle of embodiment 1, wherein a mean diameter of the hydrogel microparticle is about 0.1 μm to about 200 μm.
Embodiment 5. The hydrogel microparticle of embodiment 1, wherein the N-terminus adapter is maleimide hexanoate, cysteine, or 3-[(3-acrylamidepropyl)dimethylammonio]propanoate.
Embodiment 6. The hydrogel microparticle of embodiment 1, wherein the side chain adapter is a zwitterionic phosphoryl choline moiety.
Embodiment 7. The hydrogel microparticle of embodiment 1, wherein the at least one peptide comprises a linker between the N-terminus adapter and the peptide.
Embodiment 8. The hydrogel microparticle of embodiment 7, wherein the linker is C₆ or Gly-Gly.
Embodiment 9. The hydrogel microparticle of embodiment 1, wherein the at least one polysaccharide has a concentration in the hydrogel microparticle of about 5.0% to about 50%.
Embodiment 10. The hydrogel microparticle of embodiment 1, further comprising a fluorescent probe.
Embodiment 11. The hydrogel microparticle of embodiment 1, wherein the florescent probe is fluorescein.
Embodiment 12. A method for screening of a peptide for immunogenic activity or tolerogenic activity, the method comprising:
a) co-culturing the hydrogel of embodiment 1 with an immune cell;
b) measuring NFκB activity, CCR7 activity, CD40 activity, or any combination thereof in the immune cell; and
c) identifying the peptide with immunogenic activity when the NFκB activity, CCR7 activity, or CD40 activity is at least about 0.01% to about 500% than the NFκB activity, CCR7 activity, or CD40 activity of an immune cell that has not been co-cultured with the hydrogel of claim 1.
Embodiment 13. The method of embodiment 12, wherein the immune cell is a dendritic cell line or a macrophage cell line.
Embodiment 14. A method for associating a barcode with gene expression in a cell, the method comprising:
a) co-culturing the hydrogel of embodiment 1 with a cell;
b) loading an indexing primer with the hydrogel and the cell, wherein the indexing primer comprises a barcode sequence and hybridizes with an mRNA transcript of a gene in the cell;
c) reverse transcribing the mRNA transcript to yield a cDNA sequence;
d) amplifying the cDNA sequence to yield an amplified cDNA sequence; and
e) sequencing the amplified cDNA sequence.

### REFERENCES

[1] Ulrich, C., Bichel, J., Euvrard, S., Guidi, B., Proby, C. M., Van De Kerkhof, P. C. M., Amerio, P., Rønnevig, J., Slade, H. B., & Stockfleth, E., “Topical immunomodulation under systemic immunosuppression: Results of a multicentre, randomized, placebo-controlled safety and efficacy study of imiquimod 5% cream for the treatment of actinic keratoses in kidney, heart, and liver transplant patients,” Br. J. Dermatol., vol. 157, no. SUPPL. 2, pp. 25-31, December 2007.
[2] Cornejo-Garcia, J. A., Perkins, J. R., Jurado-Escobar, R., Garcia-Martin, E., Agúndez, J. A., Viguera, E., Pérez-Siáchez, N., & Blanca-López, N., “Pharmacogenomics of prostaglandin and leukotriene receptors,” Front. Pharmacol., vol. 7, no. SEP, September 2016.
[3] Flores, C., Fouquet, G., Moura, I. C., Maciel, T. T., & Hermine, O., “Lessons to learn from low-dose cyclosporin-A: A new approach for unexpected clinical applications,” Front. Immunol., vol. 10, no. MAR, 2019.
[4] Ahlmann, M. & Hempel, G., “The effect of cyclophosphamide on the immune system: implications for clinical cancer therapy,” Cancer Chemother. Pharmacol., vol. 78, no. 4, pp. 661-671, October 2016.
[5] Jantan, I., Ahmad, W., & Bukhari, S. N. A., “Plant-derived immunomodulators: An insight on their preclinical evaluation and clinical trials,” Front. Plant Sci., vol. 6, no. AUG, August 2015.
[6] Kang, H. K., Lee, H. H., Seo, C. H., & Park, Y., “Antimicrobial and Immunomodulatory Properties and Applications of Marine-Derived Proteins and Peptides,” Mar. Drugs, vol. 17, no. 6, June 2019.
[7] Demento, S. L., Siefert, A. L., Bandyopadhyay, A., Sharp, F. A., & Fahmy, T. M., “Pathogen-associated molecular patterns on biomaterials: a paradigm for engineering new vaccines,” Trends Biotechnol., vol. 29, no. 6, pp. 294-306, June 2011.
[8] Kim, S. H., Yang, I. Y., Kim, J., Lee, K. Y., & Jang, Y. S., “Antimicrobial peptide LL-37 promotes antigen-specific immune responses in mice by enhancing Th17-skewed mucosal and systemic immunities,” Eur. J. Immunol., vol. 45, no. 5, pp. 1402-1413, May 2015.
[9] Azuma, M., Sawahata, R., Akao, Y., Ebihara, T., Yamazaki, S., Matsumoto, M., Hashimoto, M., Fukase, K., Fujimoto, Y., & Seya, T., “The Peptide Sequence of Diacyl Lipopeptides Determines Dendritic Cell TLR2-Mediated NK Activation,” PLoS One, vol. 5, no. 9, p. e12550, 2010.
[10] Kwon, Y. W., Bae, S., Jo, Y. S., Seo, Y., & Yoon, J. H., “Stimulation of the migration and expansion of adult mouse neural stem cells by the fpr2-specific peptide wkymvm,” Life, vol. 11, no. 11, November 2021.
[11] Xiao, Y., Reis, L. A., Feric, N., Knee, E. J., Gu, J., Cao, S., Laschinger, C., Londono, C., Antolovich, J., McGuigan, A. P., et al., “Diabetic wound regeneration using peptide-modified hydrogels to target re-epithelialization,” Proc. Natl. Acad. Sci. U.S.A, vol. 113, no. 40, pp. E5792-E5801, October 2016.
[12] Cai, H., Wu, F. Y., Wang, Q. L., Xu, P., Mou, F. F., Shao, S. J., Luo, Z. R., Zhu, J., Xuan, S. S., Lu, R., et al., “Self-assembling peptide modified with QHREDGS as a novel delivery system for mesenchymal stem cell transplantation after myocardial infarction,” FASEB J., vol. 33, no. 7, pp. 8306-8320, July 2019.
[13] Wuerth, K. C., Falsafi, R., & Hancock, R. E. W., “Synthetic host defense peptide IDR-1002 reduces inflammation in Pseudomonas aeruginosa lung infection,” PLoS One, vol. 12, no. 11, p. e0187565, November 2017.
[14] Bozovic̆ar, K. & Bratkovic̆, T., “Evolving a Peptide: Library Platforms and Diversification Strategies,” Int. J. Mol. Sci., vol. 21, no. 1, January 2020.
[15] Klein, A. M., Weitz, D. A., Kirschner, M. W., Mazutis, L., Akartuna, I., Tallapragada, N., Veres, A., Li, V., & Peshkin, L., “Droplet Barcoding for Single-Cell Transcriptomics Applied to Embryonic Stem Cells Accession Numbers GSE65525 Klein et al Resource Droplet Barcoding for Single-Cell Transcriptomics Applied to Embryonic Stem Cells,” Cell, vol. 161, 2015.
[16] Yoon, Y. M., Lewis, J. S., Carstens, M. R., Campbell-Thompson, M., Wasserfall, C. H., Atkinson, M. A., & Keselowsky, B. G., “A combination hydrogel microparticle-based vaccine prevents type 1 diabetes in non-obese diabetic mice,” Sci. Rep., vol. 5, August 2015.
[17] Chen, X. Y., Butt, A. M., & Mohd Amin, M. C. I., “Enhanced paracellular delivery of vaccine by hydrogel microparticles-mediated reversible tight junction opening for effective oral immunization,” J. Control. Release, vol. 311-312, pp. 50-64, October 2019.
[18] Truong, N. F., Kurt, E., Tahmizyan, N., Lesher-Pérez, S. C., Chen, M., Darling, N. J., Xi, W., & Segura, T., “Microporous annealed particle hydrogel stiffness, void space size, and adhesion properties impact cell proliferation, cell spreading, and gene transfer,” Acta Biomater., vol. 94, pp. 160-172, August 2019.
[19] Roth, G. A., Picece, V. C. T. M., Ou, B. S., Luo, W., Pulendran, B., & Appel, E. A., “Designing spatial and temporal control of vaccine responses,” Nat. Rev. Mater. 2021 73, vol. 7, no. 3, pp. 174-195, September 2021.
[20] Liang, X., Li, L., Li, X., He, T., Gong, S., Zhu, S., Zhang, M., Wu, Q., & Gong, C., “A spontaneous multifunctional hydrogel vaccine amplifies the innate immune response to launch a powerful antitumor adaptive immune response,” Theranostics, vol. 11, no. 14, p. 6936, 2021.
[21] Roberts, R. A., Eitas, T. K., Byrne, J. D., Johnson, B. M., Short, P. J., McKinnon, K. P., Reisdorf, S., Luft, J. C., DeSimone, J. M., & Ting, J. P., “Towards programming immune tolerance through geometric manipulation of phosphatidylserine,” Biomaterials, vol. 72, pp. 1-10, December 2015.
[22] Bushnell, G. G., Rao, S. S., Hartfield, R. M., Zhang, Y., Oakes, R. S., Jeruss, J. S., & Shea, L. D., “Microporous scaffolds loaded with immunomodulatory lentivirus to study the contribution of immune cell populations to tumor cell recruitment in vivo,” Biotechnol. Bioeng., vol. 117, no. 1, p. 210, January 2020.
[23] Jayaraman, N., “Multivalent ligand presentation as a central concept to study intricate carbohydrate-protein interactions,” Chem. Soc. Rev., vol. 38, no. 12, pp. 3463-3483, November 2009.
[24] Chowdhury, S., Toth, I., & Stephenson, R. J., “Dendrimers in vaccine delivery: Recent progress and advances,” Biomaterials, vol. 280, p. 121303, January 2022.
[25] Chung, Casper; Lau, Chi Ming Laurence; Sin, Dixon; Chung, Jin Teng; Zhang, Yuzi; Chau, Ying; Yao, S., “Droplet-based microfluidic synthesis of hydrogel microparticles via click chemistry-based crosslinking for controlled release of proteins,” ACS Appl. Bio Mater., 2021.
[26] Duplantier, A. J. & van Hoek, M. L., “The human cathelicidin antimicrobial peptide LL-37 as a potential treatment for polymicrobial infected wounds,” Front. Immunol., vol. 4, no. JUL, p. 143, 2013.
[27] Kim, J., Yang, Y. L., Jeong, Y., & Jang, Y. S., “Application of Antimicrobial Peptide LL-37 as an Adjuvant for Middle East Respiratory Syndrome-Coronavirus Antigen Induces an Efficient Protective Immune Response Against Viral Infection After Intranasal Immunization,” Immune Netw., vol. 22, no. 5, October 2022.
[28] Kuroda, K., Okumura, K., Isogai, H., & Isogai, E., “The human cathelicidin antimicrobial peptide LL-37 and mimics are potential anticancer drugs,” Front. Oncol., vol. 5, no. JUN, p. 144, 2015.
[29] Kei Wu, W. K., Jao Yiu Sung, J., Fai, K. T., Tao, H. L., Jie, Z. L., Man Chu, K., & Hin Cho, C., “The host defense peptide LL-37 activates the tumor-suppressing bone morphogenetic protein signaling via inhibition of proteasome in gastric cancer cells,” Wiley Online Libr., vol. 223, no. 1, pp. 178-186, April 2010.
[30] Nijnik, A., Madera, L., Ma, S., Waldbrook, M., Elliott, M. R., Easton, D. M., Mayer, M. L., Mullaly, S. C., Kindrachuk, J., Jenssen, H., et al., “Synthetic cationic peptide IDR-1002 provides protection against bacterial infections through chemokine induction and enhanced leukocyte recruitment,” J. Immunol., vol. 184, no. 5, pp. 2539-2550, March 2010.
[31] Miklas, J. W., Dallabrida, S. M., Reis, L. A., Ismail, N., Rupnick, M., & Radisic, M., “QHREDGS Enhances Tube Formation, Metabolism and Survival of Endothelial Cells in Collagen-Chitosan Hydrogels,” PLoS One, vol. 8, no. 8, August 2013.
[32] Mandla, S., Davenport Huyer, L., Wang, Y., & Radisic, M., “Macrophage Polarization with Angiopoietin-1 Peptide QHREDGS,” ACS Biomater. Sci. Eng., vol. 5, no. 9, pp. 4542-4550, September 2019.
[33] Baffert, F., Le, T., Thurston, G., & McDonald, D. M., “Angiopoietin-1 decreases plasma leakage by reducing number and size of endothelial gaps in venules,” Am. J. Physiol.—Hear. Circ. Physiol., vol. 290, no. 1, January 2006.
[34] Schaefer, A. K., Melnyk, J. E., He, Z., Del Rosario, F., & Grimes, C. L., “Pathogen- and Microbial-Associated Molecular Patterns (PAMPs/MAMPs) and the Innate Immune Response in Crohn's Disease,” Immun. Inflamm. Heal. Dis. Emerg. Roles Nutraceuticals Funct. Foods Immune Support, pp. 175-187, January 2018.
[35] Elson, C. O. & Dertzbaugh, M. T., “Mucosal Adjuvants,” Mucosal Immunol. Two-Volume Set, pp. 967-986, January 2005.
[36] Adam, A., Petit, J. F., Lefrancier, P., & Lederer, E., “Muramyl peptides—Chemical structure, biological activity and mechanism of action,” Mol. Cell. Biochem., vol. 41, no. 1, pp. 27-47, December 1981.
[37] Kawai, Y., Akagawa, K., & Yano, I., “Lipoamino acids which are similar to bacterial endotoxin in both structure and biological activity related to physiological function,” Adv. Exp. Med. Biol., vol. 256, pp. 159-162, 1990.
[38] Dong, Y., Love, K. T., Dorkin, J. R., Sirirungruang, S., Zhang, Y., Chen, D., Bogorad, R. L., Yin, H., Chen, Y., Vegas, A. J., et al., “Lipopeptide nanoparticles for potent and selective siRNA delivery in rodents and nonhuman primates,” Proc. Natl. Acad. Sci. U.S.A, vol. 111, no. 11, pp. 3955-3960, March 2014.
[39] Horváth, A., Olive, C., Wong, A., Clair, T., Yarwood, P., Good, M., & Toth, I., “Lipoamino acid-based adjuvant carrier system: Enhanced immunogenicity of group A streptococcal peptide epitopes,” J. Med. Chem., vol. 45, no. 6, pp. 1387-1390, March 2002.
[40] Li, B., Yuan, Z., Jain, P., Hung, H. C., He, Y., Lin, X., McMullen, P., & Jiang, S., “De novo design of functional zwitterionic biomimetic material for immunomodulation,” Sci. Adv., vol. 6, no. 22, May 2020.

1. A hydrogel microparticle comprising at least one polysaccharide and at least one peptide, wherein the at least one peptide comprises an N-terminus adapter or a side chain adapter.

2. The hydrogel microparticle of claim 1, wherein the at least one polysaccharide is dextran.

3. The hydrogel microparticle of claim 1, wherein the at least one peptide comprises the amino acid sequence FKRIVQRIKDFLR (SEQ ID NO: 1), VQRWLIVWRIRK (SEQ ID NO: 2), QHREDGS (SEQ ID NO: 3), CSKKKK (SEQ ID NO: 4), SKKKK (SEQ ID NO: 5), GGCSKKKK (SEQ ID NO: 6), RGPPP (SEQ ID NO: 7), GG, GA, GV, GL, GF, GC, GD, GK, S, T, or Y.

4. The hydrogel microparticle of claim 1, wherein a mean diameter of the hydrogel microparticle is about 0.1 μm to about 200 μm.

5. The hydrogel microparticle of claim 1, wherein the N-terminus adapter is maleimide hexanoate, cysteine, or 3-[(3-acrylamidepropyl)dimethylammonio]propanoate.

6. The hydrogel microparticle of claim 1, wherein the side chain adapter is a zwitterionic phosphoryl choline moiety.

7. The hydrogel microparticle of claim 1, wherein the at least one peptide comprises a linker between the N-terminus adapter and the peptide.

8. The hydrogel microparticle of claim 7, wherein the linker is C₆ or Gly-Gly.

9. The hydrogel microparticle of claim 1, wherein the at least one polysaccharide has a concentration in the hydrogel microparticle of about 5.0% to about 50%.

10. The hydrogel microparticle of claim 1, further comprising a fluorescent probe.

11. The hydrogel microparticle of claim 1, wherein the florescent probe is fluorescein.

12. A method for screening of a peptide for immunogenic activity or tolerogenic activity, the method comprising:
a) co-culturing the hydrogel of claim 1 with an immune cell;
b) measuring NFκB activity, CCR7 activity, CD40 activity, or any combination thereof in the immune cell; and
c) identifying the peptide with immunogenic activity when the NFκB activity, CCR7 activity, or CD40 activity is at least about 0.01% to about 500% greater than the NFκB activity, CCR7 activity, or CD40 activity of an immune cell that has not been co-cultured with the hydrogel of claim 1.

13. The method of claim 12, wherein the immune cell is a dendritic cell line or a macrophage cell line.

14. A method for associating a barcode with gene expression in a cell, the method comprising:
a) co-culturing the hydrogel of claim 1 with a cell;
b) loading an indexing primer with the hydrogel and the cell, wherein the indexing primer comprises a barcode sequence and hybridizes with an mRNA transcript of a gene in the cell;
c) reverse transcribing the mRNA transcript to yield a cDNA sequence;
d) amplifying the cDNA sequence to yield an amplified cDNA sequence; and
e) sequencing the amplified cDNA sequence.


SEQUENCE LISTING (14 sequences)

SEQ ID NO: 1 - protein, 13 aa, synthetic construct
FKRIVQRIKDFLR

SEQ ID NO: 2 - protein, 12 aa, synthetic construct
VQRWLIVWRIRK

SEQ ID NO: 3 - protein, 7 aa, synthetic construct
QHREDGS

SEQ ID NO: 4 - protein, 6 aa, synthetic construct
CSKKKK

SEQ ID NO: 5 - protein, 5 aa, synthetic construct
SKKKK

SEQ ID NO: 6 - protein, 8 aa, synthetic construct
GGCSKKKK

SEQ ID NO: 7 - protein, 5 aa, synthetic construct
RGPPP

SEQ ID NO: 8 - protein, 37 aa, synthetic construct
LLGDFFRKSKEKIGKEFKRIVQRIKDFLRNLVPRTES

SEQ ID NO: 9 - DNA, 22 bp, synthetic construct
  source 1..22: mol_type = other DNA
aaggcgtcacaagcaatcactc

SEQ ID NO: 10 - DNA, 24 bp, synthetic construct
  source 1..24: mol_type = other DNA
gaagagcgtcgtgtagggaaagag

SEQ ID NO: 11 - DNA, 20 bp, synthetic construct
  source 1..20: mol_type = other DNA
tttttttttttttttttttv

SEQ ID NO: 12 - protein, 4 aa, synthetic construct
EAAK

SEQ ID NO: 13 - protein, 4 aa, synthetic construct
GTAR

SEQ ID NO: 14 - protein, 4 aa, synthetic construct
DRIR